(19) United States
(12) Patent Application Publication
(10) Pub. No.: US 2008/0206182 A1
(43) Pub. Date: Aug. 28, 2008

(54) Conjugates of a Polymer and a Protein Linked by an Oxime Group

(75) Inventors: Klaus Sommermeyer, Rosbach (DE); Ronald Frank, Meine-Grassel (DE); Norbert Zander, Meine (DE)

Correspondence Address: FISH & RICHARDSON P.C., PO BOX 1022, MINNEAPOLIS, MN, 55440-1022, US

(73) Assignee: FRESENIUS KABI DEUTSCHLAND GMBH, Bad Homburg v.d.H. (DE)

(21) Appl. No.: 10/567,265

(22) PCT Filed: Aug. 6, 2004
(86) PCT No.: PCT/EP2004/008820

§ 371 Date: Apr. 10, 2008

Related U.S. Application Data
(60) Provisional application No. 60/552,157, filed on Mar. 11, 2004.

(30) Foreign Application Priority Data
Aug. 8, 2003 (EP) PCT/EP03/08829
Aug. 8, 2003 (EP) PCT/EP03/08858
Aug. 8, 2003 (EP) PCT/EP03/08859
Mar. 11, 2004 (EP) 04005872.9

(51) Int. Cl. A61K 38/18; C07K 2/00; C08F 226/10; C08F 16/06; C08G 67/00; C08B 31/00; C08B 37/02; C07K 14/475; C07K 14/56; C07K 14/53; A61K 38/19

(52) U.S. Cl. 424/78.17; 525/54.1; 525/326.7; 525/56; 525/415; 525/410; 536/123.1; 536/112; 536/102; 525/374

(57) ABSTRACT

The present invention relates to polymers functionalized by an aminooxy group or a derivative thereof, conjugates, wherein the functionalized polymers are covalently coupled with a protein by an oxime linking group, a process for preparing the functionalized polymers, a process for preparing the conjugates, functionalize polymers as obtainable by the process of the present invention, conjugates as obtainable by the process of the present invention, and pharmaceutical compositions comprising at least one conjugate of the present invention and the use of said conjugates and compositions for the prophylaxis or therapy of the human or animal body.

The present invention relates to polymers functionalized by an aminooxy group or a derivative thereof, conjugates, wherein the functionalized polymers are covalently coupled with a protein by an oxime linking group, a process for preparing the functionalized polymers, a process for preparing the conjugates, functionalized polymers as obtainable by the process of the present invention, conjugates as obtainable by the process of the present invention, and pharmaceutical compositions comprising at least one conjugate of the present invention and the use of said conjugates and compositions for the prophylaxis or therapy of the human or animal body.

Covalent attachment of functionalized polymers like polysaccharides, for example starch and derivatives thereof, and dextrane and derivatives thereof and polyethylene glycol and derivatives thereof to therapeutic proteins prolongs the circulatory life time of the proteins in vivo, reduces their antigenicity and immunogenicity, and improves their resistance to proteolysis. These properties are of great clinical interest, especially in the case of relatively small proteins, where it is believed that an increase of Stoke's radius is consistent with a reduced renal clearance.

Modifications of proteins with biocompatible polymers are known in the art. So far most of the modifications of proteins have been carried out by using polyethylene glycol or dextran, whereby polyethylene glycol (PEG) has been preferably used. Further, modifications of proteins with other polysaccharides are known in the art.

WO 02/09766 discloses, among others, biocompatible protein-polymer compounds which are produced by conjugation of biologically active protein with a biocompatible polymer derivative. The biocompatible polymers used are highly reactive branched polymers, and the resulting conjugates contain a long linker between polymer derivative and protein. As biocompatible polymers, polymers of formula (P—OCH2CO—NH—CHR—CO—)n-L-Qk-A are described, wherein P and Q are polymeric residues and k may be 1 or 0. For P and Q, polyethylene glycol, polypropylene glycol, polyoxyethylene, polytrimethylene glycol, polylactic acid and its derivatives, polyacrylic acid and its derivatives, polyamino acid, polyvinyl alcohol, polyurethane, polyphosphazene, poly(L-lysine), polyalkylene oxide, polyacryl amide and water soluble polymers such as dextran or polysaccharide are mentioned. As proteins, among others, alpha, beta and gamma interferons, blood factors, cytokines such as interleukins, G-CSF, GM-CSF are mentioned. In the examples of WO 02/09766, only mono-, di- and tri-polyethyleneglycol derivatives are disclosed which are coupled exclusively to interferon and epidermal growth factor, and human growth hormone.

WO 94/01483 discloses biocompatible polymer conjugates which are formed by covalently binding a biologically inactive polymer or polymer derivative to a pharmaceutically pure, synthetic hydrophilic polymer via specific types of chemical bonds. As naturally occurring polymers and derivatives thereof, polysaccharides such as hyaluronic acid, proteoglycans such as chondroitin sulfates A, B and C, chitin, heparin, heparin sulfate, dextranes such as cyclodextrane, hydroxyethyl cellulose, cellulose ether and starch, lipids such as triglycerides and phospholipids are disclosed. As synthetic polymers, among others, polyethylene and derivatives thereof are described having an average molecular weight of from about 100 to about 100,000. As proteins linked to the polymer or the polymer derivative, cytokines and growth factors are described, including interferons, tumor necrosis factors, interleukins, colony stimulating factors, growth factors such as osteogenic factor extract, epidermal growth factor, transforming growth factor, platelet derived growth factor, acidic fibroblast growth factor and others are disclosed. In all working examples of WO 94/01483, polyethylene glycols derivatives are used as polymer.

WO 96/11953 discloses N-terminally chemically modified protein compounds and methods of their production. Specifically, G-CSF compositions are described which result from coupling a water soluble polymer to the N terminus of G-CSF. In the context of WO 96/11953, also consensus interferone N-terminally coupled to water soluble polymers are disclosed. While a wide variety of water soluble polymers are listed in WO 96/11953 (e.g. copolymers of ethylene glycol and propylene glycol, carboxymethyl cellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), poly(n-vinyl pyrrolidone)polyethylene glycol, polypropylene glycol homopolymers, polypropylene oxide/ethylene oxide copolymers or polyoxyethylated polyols), only PEGylated G-CSF or consensus IFN compositions are described in the working examples of WO 96/11953.

WO 97/30148 relates to polypeptide conjugates with reduced allergenicity comprising a polymeric carrier molecule having two or more polypeptide molecules coupled thereto. These conjugates are preferably part of compositions used in the personal care market. Said conjugates are produced by activating a polymeric carrier molecule, reacting two or more polypeptide molecules with the activated polymeric carrier molecule and blocking of residual active groups on the conjugate. As polymeric carrier molecule, a vast variety is listed in WO 97/30148, including such different groups of compounds like natural or synthetic homopolymers such as polyols, polyamines, polycarboxylic acids and heteropolymers comprising at least two different attachment groups. Examples are given, which comprise star PEGs, branched PEGs, polyvinyl alcohols, polycarboxylates, polyvinylpyrrolidones and poly-D,L-amino acids. Among others, also dextrans such as carboxymethyl dextran, celluloses such as hydroxyethyl cellulose or hydroxypropyl cellulose, hydrolysates of chitosan, starches such as hydroxyethyl starches or hydroxypropyl starches, glycogen, agarose, guar gum, inulin, pullulan, xanthan gum, carrageenin, pectin, alginic acid etc. are disclosed. As polypeptides, only some enzymes are explicitly disclosed.

Baldwin, J. E. et al., Tetrahedron, vol. 27 (1981), pp. 1723-1726 describe the chemical modification of dextran and hydroxyethyl starch to give aldehyde substituted polymers which are allowed to react with hemoglobin to give soluble polymer-bound hemoglobins. These were shown to be capable of binding oxygen, but heart perfusion experiments clearly indicated that the polymer-bound hemoglobins were not suitable for use as blood substitutes.

WO 99/49897 describes conjugates of hemoglobin formed by reacting polysaccharides such as dextrane or hydroxyethyl starch with amino groups of the hemoglobin. As functional groups of the polysaccharide, aldehyde groups produced by oxidative saccharide ring-opening are used. As preferred reducing agent used, borane dimethylamine is disclosed. Moreover, WO 99/49897 is exclusively limited to hemoglobin.

WO 03/074087 relates to a method of coupling proteins to a starch-derived modified polysaccharide. The binding action between the protein and the polysaccharide, hydroxyalkyl starch, is a covalent linkage which is formed between the terminal aldehyde group or a functional group resulting from chemical modification of said terminal aldehyde group of the hydroxy alkyl starch molecule, and a functional group of the protein. As reactive group of the protein, amino groups, thio groups and carboxyl groups are disclosed, and aldehyde groups of the protein are not mentioned. Moreover, while a vast variety of possibilities of different linkages is given in the form of many lists, including different functional groups, theoretically suitable different linker molecules, and different chemical procedures, the working examples describe only two alternatives: first, an oxidized hydroxyethyl starch is used and coupled directly to proteins using ethyldimethylaminopropyl carbodiimide (EDC) activation, or a non-oxidized hydroxyethyl starch is used and coupled directly to a protein forming a Schiff's base which is subsequently reduced to the respective amine.

Gaertner et al., Bioconjugate Chem. 1996, 7, 38-44 discloses a site specific attachment of functionalized polyethylene glycol (PEG) to the amino terminus of proteins. The PEG and the protein are linked by an —NHCO—CH₂—O—N═CH— linking group. It is an object of the present invention to provide conjugates of a functionalized polymer which is covalently linked with a protein to improve the circulatory life time in vivo of the proteins employed and to reduce the antigenicity and immunogenicity of the proteins.

This object is achieved by a functionalized polymer of formula I

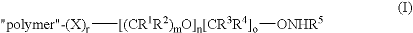

wherein the symbols have the following meanings
“polymer” soluble linear or branched homopolymers or random copolymers and derivatives thereof selected from the group consisting of alkylene glycol homopolymers, preferably ethylene glycol homopolymers (PEG), propylene glycol homopolymers, alkylene glycol copolymers, preferably propylene oxidelethylene oxide co-polymers, polyvinyl alcohol, polyvinyl pyrrolidone, poly-1,3-dioxolane, poly-1,3,6-trioxane, ethylene/maleic anhydride copolymer, polyaminoacids, and polysaccharides, preferably selected from the group consisting of starch, cellulose, dextran, gum arabic, xanthan gum, inulin, ghatti gum, pectin, guar gum, gum tragacanth, agar, algin, karaya gum, carrageenan, scleroglucan, fucellaran, arabinogalacton and locust bean gum;
R¹, R², R³, R⁴, R⁵ hydrogen, alkyl, aryl, more preferably hydrogen;
m 2 to 4, wherein the residues R¹ and R² may be the same or different in the m groups CR¹R²;
n 0 to 20, preferably 0 to 10, more preferably 1 to 5, most preferably 2;
o 0 to 20, preferably 0 to 10, more preferably 0 or 2, wherein in the case of n 0, o is not 0,
   in a preferred embodiment o is 2 to 20, preferably 2 to 10, more preferably 2, wherein the residues R³ and R⁴ may be the same or different in the o groups CR³R⁴;
r 0 or 1, preferably 1;
X —(CR⁸R⁹)ₚO—, —(CR⁸R⁹)ₚS—, —(CR⁸R⁹)ₚNR⁶—, —(CR⁸R⁹)ₚOC(O)—, —(CR⁸R⁹)ₚC(O)O—, —(CR⁸R⁹)ₚC(G)N(R¹⁰)O—, —(CR⁸R⁹)ₚN(R¹¹)O—,

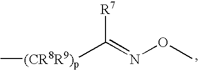

preferably

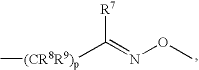

—(CR⁸R⁹)ₚC(G)N(R¹⁰)O—, —(CR⁸R⁹)ₚN(R¹¹)O—, more preferably

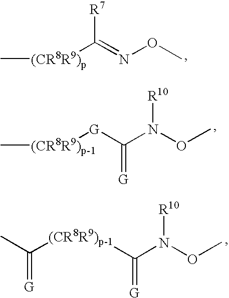

—(CR⁸R⁹)ₚN(R¹¹)O—, most preferably

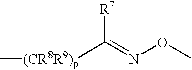

and —(CR⁸R⁹)ₚN(R¹¹)O—, wherein one or more groups —(CR⁸R⁹)— may be replaced by W, whereby a chemically reasonable group is formed;
   W O, NR¹², C(G), preferably Q, C(G);
   G S, O, NR¹⁴, preferably O;
   R⁶, R⁷, R⁸, R⁹, R¹⁰, R¹¹, R¹², R¹⁴ hydrogen, alkyl, aryl, preferably hydrogen,
   p 0 to 20, preferably 0 to 10, more preferably 0 to 5, most preferably 0 to 4, even more preferably 0 in the case of polysaccharides and derivatives thereof, and 1 to 4 in the case of polyalkylene glycols and derivatives thereof, wherein the residues R⁸ and R⁹ may be the same or different in the p groups CR⁸R⁹;
   wherein the group

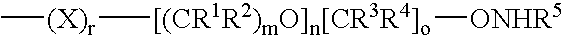

is covalently linked with least one terminal group or least one centrally located group of the “polymer”.

With regard to above-mentioned group K preferably in the case of polyalkylene glycols and derivatives thereof, two of the groups (CR⁸R⁹) may be replaced by two groups W, preferably by two groups W such that the two groups W together form a group —N(R¹²)C(G)-. In this case, X is more preferably

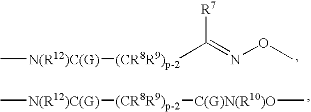

or

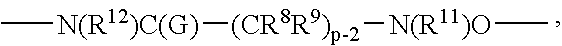

wherein p is 2, 3 or 4, preferably 3 or 4 and most preferably 4, with R⁷, R⁸, R⁹, R¹⁰, R¹¹, R¹² hydrogen, alkyl, aryl, preferably hydrogen, in a further preferred embodiment X is

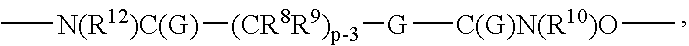

wherein p is 5, with
R⁷, R⁸, R⁹, R¹⁰, R¹¹, R¹² hydrogen, alkyl, aryl, preferably hydrogen.

With regard to above-mentioned group X, preferably in the case of polyalkylene glycols and derivatives thereof, also structures

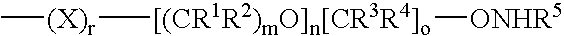

are preferred where r=1 and X=—(CR⁸R⁹)ₚO—, —N(R¹²)C(G)-(CR⁸R⁹)ₚ₋₂—S— or —(CR⁸R⁹)ₚS—, wherein in the case of —N(R²)C(G)-(CR⁸R⁹)ₚ₋₂—S— p is 2 to 20, preferably 2 to 10, more preferably 2 to 5, most preferably 2 to 4. In these cases, it is still more preferred that n=0 and o=2 to 10, preferably 2 to 8 and still more preferably 2 to 6 such as 2 or 4 or 6.

The functionalized polymers of the present invention are useful as precursors for the preparation of polymer-linker-protein conjugates.

In the context of the present invention the terms alkyl and aryl have the following meaning:

The group “alkyl” is a linear, branched or cyclic substituted or unsubstituted C1 to C20, preferably C1 to C9, more preferably C1 to C4 alkyl group. The alkyl group may be unsubstituted or substituted with aryl groups, halogen, nitro, ether, alkoxy, amino or carboxylic groups. Preferably, the alkyl groups are unsubstituted. Further, one or more non adjacent carbon atoms of the alkyl group may be replaced by hetero atoms selected from O, S and N. The hetero atoms are optionally substituted with hydrogen, allyl as mentioned above or aryl in accordance with their valency. Preferred alkyl groups are methyl, ethyl, iso-propyl, n-propyl, iso-butyl, n-butyl, sec-butyl and tert-butyl.

The group “aryl” is preferably a C6-aryl group. The aryl group may be unsubstituted or substituted with linear, branched or cyclic alkyl groups as mentioned above. The aryl group may further contain one or more hetero atoms in the ring, preferably selected from N, O and S. Most preferably the allyl group is phenyl or toluoyl.

“polymer”

Suitable “polymer”s are polysaccharides, preferably selected from the group consisting of starch, cellulose, dextran, gum arabic, xanthan gum, inulin, ghatti gum, pectin, guar gum, gum tragacanth, agar, algin, karaya gum, carrageenan, scleroglucan, fucellaran, arabinogalacton and locust bean gum. In the context of the present invention “polysaccharides” means polysaccharides as mentioned above as well as derivatives thereof. Derivatives of the polysaccharides mentioned above and the preparation thereof as well as the polysaccharides mentioned above are known by a person skilled in the art. Preferred polysaccharides are selected from the group consisting of starch, preferably hydroxyalkyl starch (HAS), and dextran.

In the context of the present invention, the term “hydroxyalkyl starch” (HAS) refers to a starch derivative which has been substituted by at least one hydroxyalkyl group. A preferred hydroxyalkyl starch of the present invention has a constitution according to formula (VI)

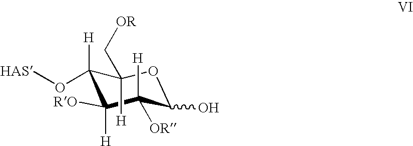

wherein the reducing end of the starch molecule is shown in the non-oxidized form and the terminal saccharide unit is shown in the hemiacetal form which, depending on e.g. the solvent, may be in equilibrium with the aldehyde form. The abbreviation HAS as used in the context of the present invention refers to the HAS molecule without the terminal saccharide unit at the reducing end of the HAS molecule.

The term hydroxyalkyl starch as used in the present invention is not limited to compounds where the terminal carbohydrate moiety comprises hydroxyalkyl groups R, R′, and/or R″ as depicted, for the sake of brevity, in formula (VI), but also refers to compounds in which at least one hydroxyalkyl group is present anywhere, either in the terminal carbohydrate moiety and/or in the remaining part of the starch molecule, HAS′, is substituted by a hydroxyalkyl group R, R′, or R″.

Hydroxyalkyl starch comprising two or more different hydroxyalkyl groups are also possible.

The at least one hydroxyalkyl group comprised in HAS may contain two or more hydroxy groups. According to a preferred embodiment, the at least one hydroxyalkyl group comprised HAS contains one hydroxy group.

The expression “hydroxyalkyl starch” also includes derivatives wherein the alkyl group is mono- or polysubstituted. In this context, it is preferred that the alkyl group is substituted with a halogen, especially fluorine, or with an aryl group. Furthermore, the hydroxy group of a hydroxyalkyl group may be esterified or etherified.

Furthermore, instead of alkyl, also linear or branched substituted or unsubstituted alkene groups may be used.

Hydroxyalkyl starch is an ether derivative of starch. Besides of said ether derivatives, also other starch derivatives can be used in the context of the present invention. For example, derivatives are useful which comprise esterified hydroxy groups. These derivatives may be e.g. derivatives of unsubstituted mono- or dicarboxylic acids with 2-12 carbon atoms or of substituted derivatives thereof. Especially useful are derivatives of unsubstituted monocarboxylic acids with 2-6 carbon atoms, especially derivatives of acetic acid. In this context, acetyl starch, butyl starch and propyl starch are preferred.

Furthermore, derivatives of unsubstituted dicarboxylic acids with 2-6 carbon atoms are preferred.

In the case of derivatives of dicarboxylic acids, it is useful that the second carboxy group of the dicarboxylic acid is also esterified. Furthermore, derivatives of monoalkyl esters of dicarboxylic acids are also suitable in the context of the present invention.

For the substituted mono- or dicarboxylic acids, the substitute groups may be preferably the same as mentioned above for substituted alkyl residues.

Techniques for the esterification of starch are known in the art (see e.g. Klerur D. et al, Comprehensive Cellulose Chemistry Vol. 2, 1998, Whiley-VCH, Weinheim, N.Y., especially chapter 4.4, Esterification of Cellulose (ISBN 3-527-29489-9).

According to a preferred embodiment of the present invention, hydroxyalkyl starch according to above-mentioned formula (VI) is employed. In formula (VI), the saccharide ring described explicitly and the residue denoted as HAS′ together represent the preferred hydroxyalkyl starch molecule. The other saccharide ring structures comprised in HAS′ may be the same as or different from the explicitly described saccharide ring.

As far as the residues R, R′ and R″ according to formula (VI) are concerned there are no specific limitations. According to a preferred embodiment, R, R′ and R″ are independently hydrogen or a hydroxyalkyl group, a hydroxyaryl group, a hydroxyaralkyl group or a hydroxyalkaryl group having of from 2 to 10 carbon atoms in the respective alkyl residue. Hydrogen and hydroxyallyl groups having of from 2 to 10 are preferred. More preferably, the hydroxyalkyl group has from 2 to 6 carbon atoms, more preferably from 2 to 4 carbon atoms, and even more preferably 2 carbon atoms. “Hydroxyalkyl starch” therefore preferably comprises hydroxyethyl starch, hydroxypropyl starch and hydroxybutyl starch, wherein hydroxyethyl starch and hydroxypropyl starch are particularly preferred and hydroxyethyl starch is most preferred.

The alkyl, aryl, aralkyl and/or alkaryl group may be linear or branched and suitably substituted.

Thus, R, R′ and R″ preferably may be hydroxyhexyl, hydroxypentyl, hydroxybutyl, hydroxypropyl such as 2-hydroxypropyl, 3-hydroxypropyl, 2-hydroxyisopropyl, hydroxyethyl such as 2-hydroxyethyl, hydrogen and the 2-hydroxyethyl group being especially preferred.

Hydroxyethyl starch (HES) is most preferred for all embodiments of the present invention concerning starch.

Hydroxyethyl starch (HES) is a derivative of naturally occurring amylopectin and is degraded by alpha-amylase in the body. HES is a substituted derivative of the carbohydrate polymer amylopectin, which is present in corn starch at a concentration of up to 95% by weight. HES exhibits advantageous biological properties and is used as a blood volume replacement agent and in hemodilution therapy in the clinics (Sommermeyer et al., 1987, Krankenhauspharmazie, 8(8), 271-278; and Weidler et al., 1991, Arzneim.-Forschung/Drug Res., 41, 494-498).

Amylopectin consists of glucose moieties, wherein in the main chain alpha-1,4-glycosidic bonds are present and at the branching sites alpha-1,6-glycosidic bonds are found. The physical-chemical properties of this molecule are mainly determined by the type of glycosidic bonds. Due to the nicked alpha-1,4-glycosidic bond, helical structures with about six glucose-monomers per turn are produced. The physico-chemical as well as the biochemical properties of the polymer can be modified via substitution. The introduction of a hydroxyethyl group can be achieved via alkaline hydroxyethylation. By adapting the reaction conditions it is possible to exploit the different reactivity of the respective hydroxy group in the unsubstituted glucose monomer with respect to a hydroxyethylation. Owing to this fact, the skilled person is able to influence the substitution pattern to a limited extent.

HES is mainly characterized by the molecular weight distribution and the degree of substitution. There are two possibilities of describing the substitution degree:
1. The degree can be described relatively to the portion of substituted glucose monomers with: respect to all glucose moieties.
2. The degree of substitution can be described as the molar substitution, wherein the number of hydroxyethyl groups per glucose moiety are described.

In the context of the present invention, the degree of substitution, denoted as DS, relates to the molar substitution, as described above.

HES solutions are present as polydisperse compositions, wherein each molecule differs from the other with respect to the polymerisation degree, the number and pattern of branching sites, and the substitution pattern. HES is therefore a mixture of compounds with different molecular weight. Consequently, a particular HES solution is determined by average molecular weight with the help of statistical means. In this context, Mₙ is calculated as the arithmetic mean depending on the number of molecules. Alternatively, M_{w} (or MW), the weight mean, represents a unit which depends on the mass of the HES.

In the context of the present invention, hydroxyethyl starch may preferably have a mean molecular weight (weight mean) of from 1 to 300 kD. Hydroxyethyl starch can further exhibit a preferred molar degree of substitution of from 0.1 to 0.8 and a preferred ratio between C₂:C₆ substitution in the range of from 2 to 20 with respect to the hydroxyethyl groups.

The term “mean molecular weight” as used in the context of the present invention relates to the weight as determined according to Sommermeyer et al., 1987, Krankenhauspharmazie, 8(8), 271-278; and Weidler et al., 1991, Arzneim.-Forschung/Drug Res., 41, 494-498.

According to a preferred embodiment of the present invention, the mean molecular weight of hydroxyethyl starch employed is from 1 to 300 kD, more preferably from 2 to 200 kD, more preferably of from 4 to 130 kD, more preferably of from 4 to 70 kD.

An example for HES with a mean molecular weight of about 130 kD is Voluven® from Fresenius. Voluven® is an artificial colloid, employed, e.g., for volume replacement used in the therapeutic indication for therapy and prophylaxis of hypovolaemia. The characteristics of Voluven® are a mean molecular weight of 130,000+/−20,000 D, a molar substitution of 0.4 and a C2:C6 ratio of about 9:1.

Therefore, the present invention also relates to a method and to conjugates as described above wherein the hydroxyalkyl starch is hydroxyethyl starch having a mean molecular weight of from 4 to 70 kD.

Preferred ranges of the mean molecular weight are, e.g., 4 to 70 kD or 10 to 70 kD or 12 to 70 kD or 18 to 70 kD or 50 to 70 kD or 4 to 50 kD or 10 to 50 kD or 12 to 50 kD or 18 to 50 kD or 4 to 18 kD or 10 to 18 kD or 12 to 18 kD or 4 to 12 kD or 10 to 12 kD or 4 to 10 kD.

According to particularly preferred embodiments of the present invention, the mean molecular weight of hydroxyethyl starch employed is in the range of from more than 4 kD and below 70 kD, such as about 10 kD, or in the range of from 9 to 10 kD or from 10 to 11 kD or from 9 to 11 kD, or about 12 kD, or in the range of from 11 to 12 kD or from 12 to 13 kD or from 11 to 13 kD, or about 18 kD, or in the range of from 17 to 18 kD or from 18 to 19 kD or from 17 to 19 kD, or about 50 kD, or in the range of from 49 to 50 kD or from 50 to 51 kD or from 49 to 51 kD.

As far as the degree of substitution (DS) is concerned, DS is preferably at least 0.1, more preferably at least 0.2, more preferably at least 0.4 and more preferably at least 0.7. Preferred ranges of DS are from 0.1 to 0.8, more preferably from 0.2 to 0.8, more preferably from 0.3 to 0.8 and even more preferably from 0.4 to 0.8, still more preferably from 0.1 to 0.7, more preferably from 0.2 to 0.7, more preferably from 0.3 to 0.7 and more preferably from 0.4 to 0.7. Particularly preferred values of DS are, e.g., 0.1, 0.2, 0.3, 0.4, 0.5, 0.6, 0.7 or 0.8, with 0.2, 0.3, 0.4, 0.5, 0.6, 0.7 or 0.8 being more preferred, 0.3, 0.4, 0.5, 0.6, 0.7 or 0.8 being even more preferred, 0.4, 0.5, 0.6, 0.7 or 0.8 being still more preferred and, e.g. 0.4 and 0.7 being particularly preferred.

Particularly preferred combinations of molecular weight of the hydroxyalkyl starch, preferably hydroxyethyl starch, and its degree of substitution DS are, e.g., 10 kD and 0.4 or 10 kD and 0.7 or 12 kD and 0.4 or 12 kD and 0.7 or 18 kD and 0.4 or 18 kD and 0.7 or 50 kD and 0.4 or 50 kD and 0.7.

In a further preferred embodiment the combinations of molecular weight of the hydroxyalkyl starch, preferably hydroxyethyl starch, and its degree of substitution DS are 30 kD and 0.7 or 30 kD and 0.4.

Further preferred combinations of molecular weight of the hydroxyalkyl starch, preferably hydroxyethyl starch, and its degree of substitution DS are 10 kD and 0.8, 12 kD and 0.8, 18 kD and 0.8, 30 kD and 0.8, and 50 kD and 0.8.

As far as the ratio of C₂: C₆ substitution is concerned, said substitution is preferably in the range of from 2 to 20, more preferably in the range of from 2 to 15 and even more preferably in the range of from 3 to 12.

According to a further embodiment of the present invention, also mixtures of hydroxyethyl starches may be employed having different mean molecular weights and/or different degrees of substitution and/or different ratios of C₂:C₆ substitution. Therefore, mixtures of hydroxyethyl starches may be employed having different mean molecular weights and different degrees of substitution and different ratios of C2:C₆ substitution, or having different mean molecular weights and different degrees of substitution and the same or about the same ratio of C₂:C₆ substitution, or having different mean molecular weights and the same or about the same degree of substitution and different ratios of C₂: C₆ substitution, or having the same or about the same mean molecular weight and different degrees of substitution and different ratios of C₂:C₆ substitution, or having different mean molecular weights and the same or about the same degree of substitution and the same or about the same ratio of C₂:C₆ substitution, or having the same or about the same mean molecular weights and different degrees of substitution and the same or about the same ratio of C₂:C₆ substitution, or having the same or about the same mean molecular weight and the same or about the same degree of substitution and different ratios of C₂:C₆ substitution, or having about the same mean molecular weight and about the same degree of substitution and about the same ratio of C₂:C₆ substitution.

In different functionalized polymers and/or conjugates methods according to the present invention, different hydroxyalkyl starches, preferably different hydroxy ethyl starches and/or different hydroxyalkyl starch mixtures, preferably different hydroxyethyl starch mixtures, may be employed.

Dextran containing a backbone of D-glucose units linked predominately alpha-D (1,6) with additional 1,3 branching points according to formula (VII), is produced mainly by bacteria or synthetically.

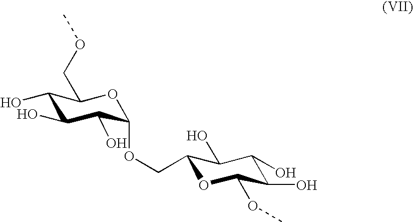

The molecular weight of the dextran is preferably from 4 kD to 300 kD, more preferably 51 kD to 100 kD and most preferably 6 kD to 40 kD. The molecular weights are determined by GPC using suitable commercially available molecular weight standards.

Further suitable polymers are soluble linear or branched polymers and derivatives thereof selected from the group consisting of alkylene glycol homopolymers, preferably ethylene glycol homopolymers (PEG) and propylene glycol homopolymers, alkylene glycol copolymers, preferably propylene oxide/ethylene oxide co-polymers, polyvinyl alcohol, polyvinyl pyrrolidone, poly-1,3-dioxolane, poly-1,3,6-trioxane, ethylene/maleic anhydride copolymer and polyaminoacids (either homopolymers or random copolymers). In the context of the present invention the polymers as mentioned above as well as derivatives thereof may be employed. The polymers mentioned above and their preparation as well as derivatives thereof are known by a person skilled in the art.

Preferred polymers are allylene glycol homopolymers, preferably ethylene glycol homopolymers (PEG) and propylene glycol homopolymers. More preferred are ethylene glycol homopolymers-(PEG).

PEGs are polyether diols of the general structure (VIII)

HO—(CH₂CH₂O)ₜ—CH₂CH₂—OH (VIM),

wherein t is the number of —(CH₂CH₂O)-groups. They are commercially available in a variety of molecular weights and low dispersity (M_{w}/Mₙ≦1.1, wherein M_{w} is the weight average molecular weight and Mₙ is the number average molecular weight). While the polyether backbone is fairly chemically inert the primary hydroxyl groups are available for derivatization. The molecular weights of PEGs used for the preparation of bioconjugates vary between 1000 and 20000 Da, although in some instances the polymers of higher and lower molecular weights than in this range are utilized. The molecular weights are determined by GPC using suitable commercially available molecular weight standards. Preferably the monoalkyl ether of PEG is employed, wherein the alkyl group is selected from a C1 to C4 allyl group. More preferably the monomethyl ether of PEG (mPEG) is used. The presence of only one derivatizable terminal group on mPEG or other monoalkyl ethers of PEG minimizes the possibilities for crosslinking and improves the homogeneity of conjugates prepared.

Besides the monoalkyl ether of PEG, preferably mPEG, other functionalized PEG derivatives may be used in the present invention. Suitable examples are halo-substituted derivatives of PEG, sulfonate esters of PEG, amino-PEG, hydrazido-PEG, mercapto-PEG, carboxyl-PEG and its active esters, aldehyde-PEG, cyanuryl chloride-PEG, and epoxide-PEG. The preparation of the derivatives mentioned above as well as further derivatives and their preparation are disclosed in S. Zalipsky, Bioconjugate Chem. 1995, 6, 150-165. Furthermore, some of the PEG derivatives are commercially available.

The functional derivatives of PEG are in general prepared by (i) direct transformation of hydroxyls to the new target functionality and (ii) reaction of the polymer with a bifunctional molecule so that one function forms an attachment to the polymer and the other one remains available for further chemical transformations.

Most preferably the monoalkyl ether of PEG, preferably mPEG, is used in the present invention.

The group X in the functionalized polymers of formula I is a linking group, selected from the group consisting of —(CR⁸R⁹)ₚO—, —(CR⁸R⁹)ₚS—, —(CR⁸R⁹)ₚNR⁶—, —(CR⁸R⁹)ₚOC(O)—, —(CR⁸R⁹)ₚC(O)O—, —(CR⁸R⁹)ₚC(G)N(R¹⁰)O—, —(CR⁸R⁹)ₚN(R¹¹)O—, and

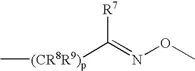

wherein one or more groups —CR⁸R⁹)— may be replaced by W, whereby a chemically reasonable group is formed.

W is O, NR¹², C(G), preferably O, C(G) and G is S, O, NR¹⁴, preferably O.

R⁶, R⁷, R⁸, R⁹, R¹⁰, R¹¹, R¹², R¹⁴ in the functionalized polymer of formula I are independently hydrogen, alkyl, aryl, preferably hydrogen.

p is 0 to 20, preferably 0 to 10, more preferably 0 to 5, most preferably 0 to 4, even more preferably O in the case of polysaccharides and derivatives thereof, and 1 to 4 in the case of polyalkylene glycols and derivatives thereof, wherein the residues R⁸ and R⁹ may be the same or different in the p groups CR⁸R⁹.

Preferred groups X are

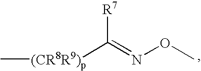

—(CR⁸R⁹)ₚC(G)N(R¹⁰)O—, and —(CR⁸R⁹)ₚN(R¹¹)O—, more preferred are

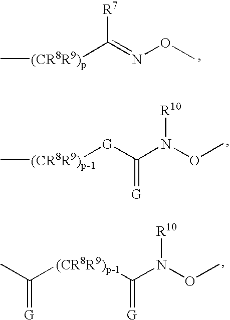

—(CR⁸R⁹)ₚN(R¹¹)O—, even more preferred are

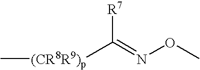

and —(CR⁸R⁹)ₚN(R¹¹)O—.

Preferred structures for the linking group X when the polymer is polyalkylene glycol or a derivative thereof are

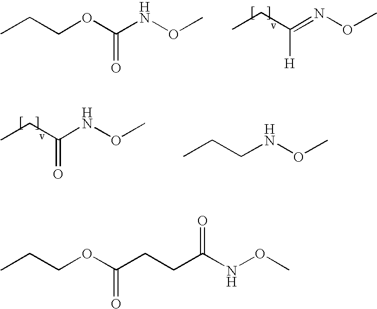

v=1 to 10, preferably 1 to 4, more preferably 2 or 3.

Most preferred is

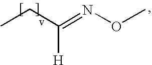

wherein v is 1 to 10, preferably 1 to 4, more preferably 2 to 3.

With regard to above-mentioned group x, in the case of polyalkylene glycols and derivatives thereof, also the following structures of the linking group X are preferred:

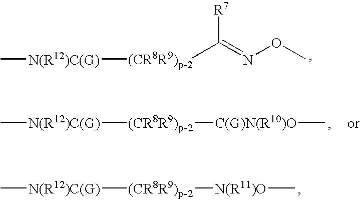

wherein p is 2, 3 or 4, preferably 3 or 4 and most preferably 4, with R⁷, R⁸, R⁹, R¹⁰, R¹¹, R¹² hydrogen, alkyl, aryl, preferably hydrogen, in a further preferred embodiment X is

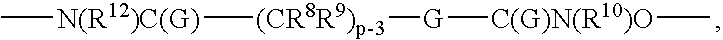

wherein p is 5, with R⁷, R⁸, R⁹, R¹⁰, R¹¹, R¹² hydrogen, alkyl, aryl, preferably hydrogen.

Even more preferred are the following structures:

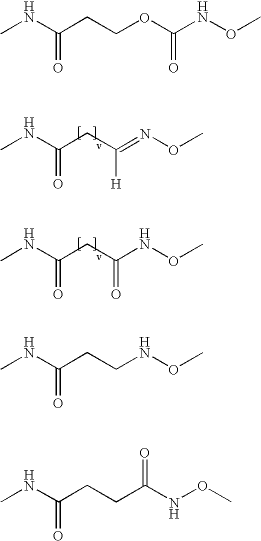

v=1 to 10, preferably 1 to 4, more preferably 2 or 3.

Most preferred are

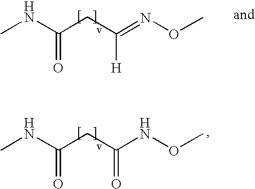

wherein v is 1 to 10, preferably 1 to 4, more preferably 2 to 3.

With regard to above-mentioned group X preferably in the case of polyalkylene glycols and derivatives thereof, also groups

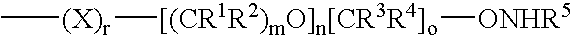

are preferred where r=1 and X=—(CR⁸R⁹)ₚO— or —(CR⁸R⁹)ₚS—,
wherein one or more groups —(CR⁸R⁹)— may be replaced by W, whereby a chemically reasonable group is formed
W is O, NR¹², C(G), preferably O, C(G) and G is S, O, NR¹⁴, preferably O;
R⁸, R⁹ and R¹² are independently hydrogen, allyl, aryl, preferably hydrogen; and
p is 2, 3 or 4.

In these cases, the following structures

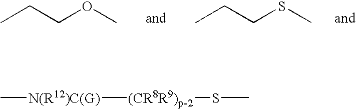

of the linking group X are especially preferred, wherein G, R⁸, R⁹, R¹² and p is 3 or 4, preferably 4.

When the polymer is a polysaccharide, preferably selected from the group consisting of dextran or a derivative thereof or starch or a derivative thereof as mentioned before, X is most preferably

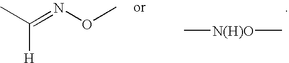

In the group —[(CR¹R²)ₘO]ₙ[CR³R⁴]ₒ— in formula I R¹, R², R³, and R⁴ are independently hydrogen, alkyl or aryl as defined above, preferably methyl or hydrogen, more preferably hydrogen. m is 2 to 4, preferably 2, wherein the residues R¹ and R² may be the same or different in the m groups CR¹R². Preferably —(CR¹R²)ₘ— is —CH₂CH₂— or —CH(CH₃)CH₂— or —CH₂CH(CH₃)—, more preferably —(CR¹R²)ₘ— is —CH₂CH₂—.

n is 0 to 20, preferably 0 to 10, more preferably 1 to 5, most preferably 1 or 2 and even more preferably 1.

o is 0 to 20, preferably 0 to 10, more preferably 0 or 2, wherein in the case of n=0, o is not 0, in a preferred embodiment o is 2 to 20, preferably 2 to 10, more preferably 2,

wherein the residues R³ and R⁴ may be the same or different in the o groups CR³R⁴.

In a most preferred embodiment of the present invention —[(CR¹R²)ₘO]ₙ[CR³R⁴]ₒ— is —CH₂CH₂OCH₂CH₂—.

In a further preferred embodiment of the present invention —[(CR¹R²)ₘO]ₙ[CR³R⁴]ₒ— is —CH₂CH₂CH(CH₃)CH₂CH₂—.

R⁵ in formula I is hydrogen, allyl, aryl as mentioned before, preferably hydrogen.

The group —[(CR¹R²)ₘO]ₙ[CR³R⁴]ₒ—ONHR⁵ in formula I is therefore in a most preferred embodiment of the present invention CH₂CH₂OCH₂CH₂—ONH₂.

The group —[(CR¹R²)ₘOₙ]ₙ[CR³R⁴]ₒ—ONHR⁵ in formula I is therefore in a further most preferred embodiment of the present invention —CH₂CH₂CH(CH₃)CH₂—ONH₂.

The group

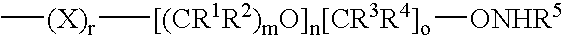

is covalently linked with least one terminal group or least one centrally located group of the “polymer”.

In general any suitable group of the polymer may be used for the covalent linkage, depending on the polymer employed. Most preferably the group

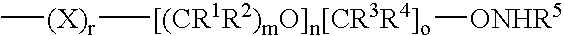

is linked with the polymer employed, preferably selected from the group consisting of starch, dextran and polyalkylene glycol or derivatives thereof, more preferably hydroxyalkyl starch, whereby hydroxyethyl starch (HES) is preferred, dextran and the monomethyl ether of polyethylene glycol (mPEG), by an oxygen comprising group of the polymer. The oxygen group may be a carbonyl group, preferably a keto group, a hemiacetal group or an aldehyde group, more preferably a hemiacetal group or an aldehyde group. In the case of polyalkylene glycols, the oxygen comprising group may also be a group OR′″″, e.g. a carboxylic acid ester or a carbonate, which is derived from the reaction of the polyalkylene glycol or a derivative thereof with alcohols, whereby preferred alcohols are selected from the group consisting of N-hydroxy succinimides such as N-hydroxy succinimide or Sulfo-N-hydroxy succinimide, suitably substituted phenols such as p-nitrophenol, o,p-dinitrophenol, o,o′-dinitrophenol, trichlorophenol such as 2,4,6-trichlorophenol or 2,4,5-trichlorophenol, trifluorophenol such as 2,4,6-trifluorophenol or 2,4,5-trifluorophenol, pentachlorophenol, pentafluorophenol, or hydroxyazoles such as hydroxy benzotriazole. Especially preferred are N-hydroxy succinimides, with N-hydroxy succinimide and Sulfo-N-hydroxy succinimide being especially preferred. However, it is also possible that the linkage with the group X is achieved by other groups than the oxygen comprising groups mentioned before. The linkage of

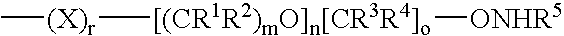

with the polymer will be explained in detail later.

The functionalized polymer of formula I is suitable for the preparation of stable conjugates with proteins.

In a further embodiment the present invention therefore relates to a conjugate of formula II

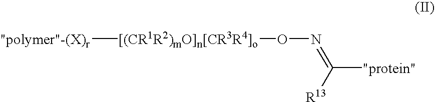

wherein the symbols “polymer”, R¹, R², R³, R⁴, m, n, o, r, X, and p have the same meaning as mentioned before and
R¹³ is hydrogen, alkyl, aryl, preferably hydrogen or methyl, and
“protein” is an amino acid sequence prepared by reaction of at least 2 amino acids
wherein in the conjugate of formula II the group

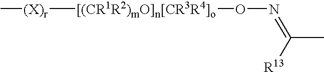

is covalently linked with least one terminal group or least one centrally located group of the “polymer” and the “protein”, preferably with an oxidized N-terminal amino acid or an oxidized carbohydrate side chain of the “protein”.

### “Protein”

The term “protein” as used in the context of the present invention, relates to any amino acid sequence having at least 2, preferably at least 5, more preferably at least 10, more preferably at least 15, more preferably at least 20, more preferably at least 25, more preferably at least 30, more preferably at least 35, more preferably at least 40, more preferably at least 45 and still more preferably at least 50 amino acids.

The protein can be produced by chemical synthetic procedures or can be of any human or another mammalian source and can be obtained by purification from naturally occurring sources.

According to the present invention, the protein can be a growth factor, a cytokine, an activator, an inhibitor, an enzyme, an antibody, an antigen, a transport protein, a bioadhesion protein, a hormone, a receptor, a suppressor, or a functional derivative or a fragment thereof. The term “functional derivative or fragment” as used in the context of the present invention relates to a derivative or fragment that maintains the desired biological property or activity of the original molecule totally or partially, e.g. at least 10%, more preferably at least 20%, more preferably at least 30%, more preferably at least 40%, more preferably at least 50%, more preferably at least 60%, more preferably at least 70%, more preferably at least 80% and especially preferably at least 90% of the desired biological property or activity of the original molecule. Particularly preferred examples of such fragments are, e.g., antibody fragments.

Examples of proteins are erythropoietin (EPO) such as recombinant human EPO (rhEPO), colony-stimulating factors (CSF), such as G-CSF like recombinant human G-CSF (rhG-CSF), alpha-Interferon (IFN alpha), beta-Interferon (IFN beta) or gamma-Interferon (IFN gamma), such as IFN alpha and IFN beta like recombinant human IFN alpha or IFN beta (rhIFN alpha or rhIFN beta), interleukines, e.g. EL-1 to IL-18 such as IL-2 or IL-3 like recombinant human IL-2 or IL-3 (rhIL-2 or rhIL-3), serum proteins such as coagulation factors II-XIII like factor VIII, factor VII, factor IX, alpha1-antitrypsin (A1AT), activated protein C (APC), plasminogen activators such as tissue-type plasminogen activator (tPA), such as human tissue plasminogen activator (hTPA), AT III such as recombinant human AT III (rhAT III), myoglobin, albumin such as bovine serum albumin (BSA), growth factors, such as epidermal growth factor (EGF), thrombocyte growth factor (PDGF), fibroblast growth factor (FGF), brain-derived growth factor (BDGF), nerve growth factor (NGF), B-cell growth factor (BCGF), brain-derived neurotrophic growth factor (BDNF), ciliary neurotrophic factor (CNTF), transforming growth factors such as TGF alpha or TGF beta, BMP (bone morphogenic proteins), growth hormones such as human growth hormone, tumor necrosis factors such as TNF alpha or TNF beta, somatostatine, somatotropine, somatomedines, hemoglobin, hormones or prohormones such as insulin, gonadotropin, melanocyte-stimulating hormone (alpha-MSM), triptorelin, hypthalamic hormones such as antidiuretic hormones (ADH and oxytocin as well as releasing hormones and release-inhibiting hormones, parathyroid hormone, thyroid hormones such as thyroxine, thyrotropin, thyroliberin, prolactin, calcitonin, glucagon, glucagon-like peptides (GLP-1, QLP-2 etc.), exendines such as exendin-4, leptin, vasopressin, gastrin, secretin, integrins, glycoprotein hormones (e.g. LH, FSH etc.), melanoside-stimulating hormones, lipoproteins and apo-lipoproteins such as apo-B, apo-E, apo-Lₙ, immunoglobulins such as IgG, IgE, IgM, IgA, IgD and fragments thereof, hirudin, tissue-pathway inhibitor, plant proteins such as lectin or ricin, bee-venom, snake-venom, immunotoxins, antigen E, alpha-proteinase inhibitor, ragweed allergen, melanin, oligolysine proteins, RGD proteins or optionally corresponding receptors for one of these proteins; or a functional derivative or fragment of any of these proteins or receptors.

Preferred enzymes are, e.g., carbohydrate-specific enzymes, proteolytic enzymes, oxidases, oxidoreductases, transferases, hydrolases, lyases, isomerases, kinases and ligases. Specific non-limiting examples are asparaginase, arginase, arginin deaminase, adenosin deaminase, glutaminase, glutaminase-asparaginase, phenylalanin, tryptophanase, tyrosinase, superoxide dismutase (SOD), endotoxinase, catalase, peroxidase, kallikrein, trypsin, chymotrypsin, elastase, thermolysin, lipase, uricase, adenosine diphosphatase, purine nucleoside phosphorylase, bilirubin oxidase, glucose oxidase, glucodase, gluconate oxidase, galactosidase, glucocerebrosidase, glucuronidase, hyaluronidase, tissue factor, streptokinase, urokinase, MAP-kinases, DNAses, RNAses, lactoferrin and functional derivatives or fragments thereof.

According to preferred embodiments of the present invention, the protein is selected from the group consisting of EPO, G-CSF, Factor VII, Factor IX, IFN beta, AT III, A1AT, Factor VIII, APC.

With the conjugates of the present invention it is intended to improve the circulatory life time in vivo of the proteins employed and to reduce the antigenicity and immunogenicity of the proteins compared to the proteins before conjunction.

In a further embodiment the present invention relates to a process for preparing a functionalized polymer comprising the step of reacting a polymer of formula III

“polymer”-(CR⁸R⁹)ₚ—Y (III)

with a compound of formula IV

Q-[(CR¹R²)ₘO]ₙ[CR³R⁴]ₒ—ONHR⁵ (IV)

wherein the symbols “polymer”, R¹, R², R³, R⁴, R⁵, m, n, o have the same meaning as mentioned before and
Y and Q are functional groups, which are suitable to react together to give one of the following linking groups —O—, —S—, —NR⁶—, —OC(O)—, —C(O)O—, —C(G)N(R¹⁰)O—, —N(R¹¹)O—,

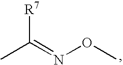

preferably

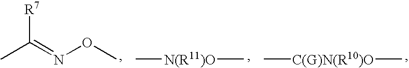

more preferably

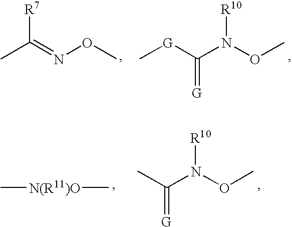

whereby in the case of

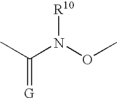

the group —(CR⁸R⁹)— is

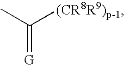

most preferably

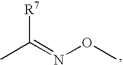

or —N(R¹¹)O—,
   in a further preferred embodiment Y and Q are functional groups, which are suitable to react together to give one of the following linking groups —O— or —S—;

wherein in the polymer of formula III one or more groups —(CR⁸R⁹)— may be replaced by W, whereby a chemically reasonable group is formed;
W is O, NR¹², C(G), preferably O, C(G);
G is S, O, NR¹⁴, preferably O;
R⁶, R⁷, R⁸, R⁹, R¹⁰, R¹¹, R¹², R¹⁴ independently hydrogen, alkyl, aryl, preferably hydrogen,
p 0 to 20, preferably 0 to 10, more preferably 0 to 5, most preferably 0 to 4, even more preferably 0 in the case of polysaccharides and derivatives thereof, and 1 to 4 in the case of polyalkylene glycols and derivatives thereof, wherein the residues R⁸ and R⁹ may be the same or different in the p groups CR⁸R⁹;
   wherein the group

(CR⁸R⁹)ₚY

is covalently linked with terminal groups or centrally located groups of the “polymer”.

Suitable groups Y are the following functional groups, among others:
C—C-double bonds or C—C-triple bonds or aromatic C—C-bonds;
the thio group or the hydroxy groups;

alkyl sulfonic acid hydrazide, aryl sulfonic acid hydrazide;
1,2-dioles;
1,2 amino-thioalcohols;
azides;
1,2-aminoalcohols;
the amino group —NH₂ or derivatives of the amino groups comprising the structure unit —NH— such as aminoalkyl groups, aminoaryl group, aminoaralkyl groups, or alkarlyaminogroups;
the hydroxylamino group —O—NH₂, or derivatives of the hydroxylamino group comprising the structure unit —O—NH—, such as hydroxylalkylamino groups, hydroxylarylamino groups, hydroxylaralkylamino groups, or hydroxalalkarylamino groups;
alkoxyamino groups, aryloxyamino groups, aralkyloxyamino groups, or alkaryloxyamino groups, each comprising the structure unit —NH—O—;
residues having a carbonyl group, -Q′-C(=G)-M, wherein G is O or S, and M is, for example,
—OH or —SH;
an alkoxy group, an aryloxy group, an aralkyloxy group, or an alkaryloxy group;
an alkylthio group, an arylthio group, an aralkylthio group, or an alkarylthio group;
an alkylcarbonyloxy group, an arylcarbonyloxy group, an aralkylcarbonyloxy group, an alkarylcarbonyloxy group;
activated esters such as esters of hydroxylamines having imid structure such as N-hydroxysuccinimide or having a structure unit O—N where N is part of a heteroaryl compound or, with G=O and Q′ absent, such as aryloxy compounds with a substituted aryl residue such as pentafluorophenyl, paranitrophenyl or trichlorophenyl;

wherein Q′ is absent or NH or a heteroatom such as S or O;
—NH—NH₂, or —NH—NH—;
—NO₂;
the nitril group;
carbonyl groups such as the aldehyde group or the keto group, or a group, which an be converted into an aldehyde group or a keto group, e.g. an acetal or hemiacetal group or a ketal or hemiketal group;
the carboxy group or an ester group;
the —N═C═O group or the —N═C═S group;
vinyl halide groups such as the vinyl iodide or the vinyl bromide group or triflate;
—C≡C—H;
—(C═NH₂Cl)-OAlkyl
groups —(C═O)—CH₂-Hal wherein Hal is Cl, Br, or I;
—CH═CH—SO₂—;
a disulfide group comprising the structure —S—S—;

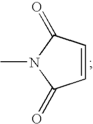

the group

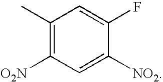

the group

A further suitable group Y is the maleimide group.

Preferred groups Y are functional groups are groups comprising a carbonyl group, preferably groups comprising an aldehyde group, a keto group, a carboxy group, an ester group, a halogen group, preferably Br or I or OTf (═O—SO₂CF₃), or a group, which an be converted into an aldehyde group or a keto group, e.g. an acetal or hemiacetal group or a ketal or hemiketal group.

Further preferred groups Y are residues having a carbonyl group, —C(=G)-M, wherein G is O, and M is an activated ester such as an ester of a hydroxylamine having imid structure such as N-hydroxysuccinimide.

In the case that the polymer is a polysaccharide, preferably selected from the group consisting of starch and derivatives thereof and dextran and derivatives thereof, Y is preferably an aldehyde group or an acetal or hemiacetal group. In this case p is preferably 0.

In the case that the polymer is a polyalkylene glycol such as a polyethylene glycol, Y is preferably an aldehyde group or an ester group, preferably a reactive ester group, such as an ester of a hydroxylamine having imid structure such as N-hydroxysuccinimide, a hydroxy group (—OH) or a thio group (—SH). More preferably, in case Y is a hydroxy group or a thio group, Q is a halogen group, preferably Br or I, or the OTf group. According to this embodiment of the present invention where X=—(CR⁸R⁹)ₚO— or —(CR⁸R⁹)ₚS—, the compound according to formula (IV) to be reacted with the polymer according to formula (III) may be employed with the functional group —ONHR⁵ in a protected form which is deprotected after reaction of (III) with (IV). As protecting group, among others, the phthalimide protecting group may be mentioned which can be removed by reacting the reaction product of (III) and (I) with hydrazine to give the terminal functional group —ONHR⁵. However, each suitable protecting which, after de-protection, results in —ONHR⁵, may be employed as well.

The polymers of formula III

“polymer”-(CR⁸R⁹)ₚ—Y (III)

are prepared by methods known by a person skilled in the art.

If polyalkylene glycols, preferably polyethylene glycol (PEG) or the monomethyl ether of PEG (mPEG), are employed, suitable methods for preparing the polymers of formula III are mentioned in S. Zalipsky, Bioconjugate Chem. 1995, 6, 150-165.

If polysaccharides, preferably starch or dextran or derivatives thereof, are employed, the polymers of formula III are polysaccharides, the group Y is preferably an aldehyde group or a hemiacetal group or an equilibrium of both.

Preferably, the aldehyde group Y in the polysaccharide is its reducing end, being in an equilibrium between an aldehyde and a hemiacetal form. If the polymer is a polysaccharide, p in the polymer of formula III is most preferably O. The linkage between the compound of formula IV and the polymer of formula II, wherein the polymer is a polysaccharide or a derivative thereof is shown in scheme 2.

The linkage between the compound of formula IV and the polymer of formula III, wherein the polymer is a polyalkylene glycol or a derivative thereof is shown in scheme 1.

To prepare the polysaccharide (“polymer”-(CR⁸R⁹)ₚ—Y of formula III) for use in the reaction with the compound of formula IV the polysaccharide may be oxidized so as to create thereon a substantial number of aldehyde groups. This can be accomplished by a variety of oxidation processes, the preferred one being reaction with a periodate (sodium odor potassium). This reaction can take place in aqueous solution at low temperature, e.g. 0 to 5° C., using an appropriate quantity of sodium periodate, chosen according to the desired degree of oxidation.

The reaction is complete in about 10 min to 4 hours. Ultrafiltration or dialysis can be used to remove undesirable low molecular weight salts and polysaccharide components, thereby offering a means of controlling the molecular weight range of oxidized polysaccharide to be reacted with the compound of formula IV. The oxidized polysaccharide can be used directly or is suitably recovered, e.g. by lyophilization, and redissolved for the reaction with the compound of formula IV.

However, it is preferred to employ the polysaccharide or a derivative thereof whereby preferred polysaccharides are mentioned before, without treatment of the polysaccharide by oxidation. The inventors found that no prior treatment of the polysaccharide is necessary, when a compound of formula IV, especially a preferred compound of formula IV as mentioned before, is employed,

Suitable groups Q are also functional groups as mentioned above, whereby the functional groups Y and Q are chosen in a way that one of the following group is obtained.

—O—, —S—, —NR⁶—, —OC(O)—, —C(O)O—, —C(G)N(R¹⁰)O—, —N(R¹¹)O,

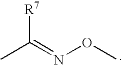

Q is preferably H₂N—O.

The group Y of the polymer of formula if is therefore preferably a group which is reactable with the group —O—NH₂. Preferred groups Y are therefore aldehyde groups, keto groups, carboxy groups, carbonate groups, and activated carboxy groups, for example ester groups, lactone groups, and amide groups. Further suitable groups are halide and pseudo halide groups and the like, for example Cl, Br, I, and OTf. Most preferably, Y is an aldehyde group (being in the case of polysaccharides—in equilibrium with a hemiacetal form). In a further preferred embodiment Y is an activated ester group such as an ester of a hydroxylamine having imid structure such as N-hydroxysuccinimide.

If Q is H₂N—O— the compounds of formula IV

Q-[(CR¹R²)ₘO]ₙ[CR³R⁴]ₒ—ONHR⁵ (IV)

are most preferably

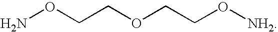

According to another embodiment of the present invention, a compound according to formula (IV) having the structure

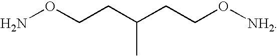

is also preferred if Q is H₂N—O—.

As examples of compounds having two —O—NH₂ groups, also compounds such as

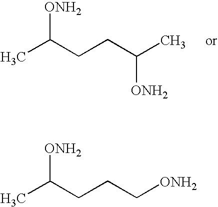

may be mentioned.

If Q is a halogen group, preferably Br or I, or the OTf group, most preferably Br or I, the compounds of formula IV

Q-[(CR¹R²)ₘ]Oₙ[CR³R⁴]ₒ—ONHR (IV)

are most preferably

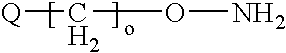

with o=2 to 10, more preferably 2 to 8 and especially preferably 2 to 6 such as 2, 4 or 6, and where —O—NH₂ may be present in its protected form such as protected with the phthalimide protecting group such that, for example, the compound according to formula (IV) may be

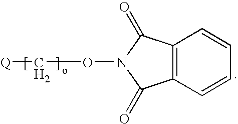

The preparation of compounds of formula (IV) of a structure as shown above, wherein o is 2-6 and Q is Br is described in Bauer et al., J. Org. Chem. 1963, 28, p. 1604. Other compounds of formula (IV) having a structure as shown above are for example obtained by an analogous method.

In a further embodiment the present invention therefore relates to a process as mentioned above, wherein the compound of formula IV is

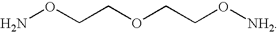

In yet a further embodiment the present invention therefore relates to a process as mentioned above, wherein the compound of formula IV is

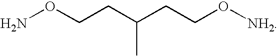

In yet a further embodiment the present invention therefore relates to a process as mentioned above, wherein the compound of formula IV is

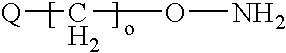

with o 2 to 10, more preferably 2 to 8 and especially preferably 2 to 6 such as 2, 4 or 6, and where —O—NH₂ may be present in its protected form.

The compounds of formula IV are prepared by methods known by a person skilled in the art. The preparation of

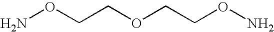

is for example disclosed in D. Boturyn et al. Tetrahedron 53 (1997) 5485-5492. Other compounds of formula IV are for example obtained by an analogous method.

For example, as to the synthesis of the compound

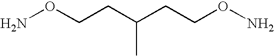

reference is made to the respective example hereinunder.

In a preferred embodiment of the process of the present invention for the preparation of functionalized polymers, the polymer of formula III is dissolved in an organic solvent, for example dichloromethane, dimethylformamide, or dimethylacetamide, or in an aqueous system, for example in a sodium acetate buffered aqueous system of pH 4.5 to 9, preferably 5 to 8. The compound of formula IV is added to the solution of the polymer. The addition is usually carried out at a temperature of from 0 to 80° C., preferably 0 to 60° C., more preferably 20 to 40° C. The mixture is agitated by stirring or shaking at said temperature usually for 1 to 48 h, preferably 2 to 24 h, more preferably 2 to 16 h, when the reaction is carried out in an organic solvent and usually for 1 to 48 h, preferably 2 to 24 h, more preferably 2 to 16 h, when the reaction is carried out in an aqueous system. The product is precipitated by adding a solvent or a solvent mixture, wherein the product is insoluble or has a low solubility. Suitable solvents for precipitation of the product depend on the nature of the product. In one embodiment the product is precipitated by adding an alcohol, preferably 2-propanol or ethanol, and incubation at a temperature usually of from −60 to 20° C., preferably −20 to 20° C. In a further embodiment the product is precipitated by a mixture of an alcohol with a low boiling polar organic solvent, for example acetone. A suitable solvent mixture is ethanol and acetone, for example a 1:1 mixture of ethanol and acetone, indicating equal volumes of said solvents, and incubation at a temperature usually of from −60 to 20° C., preferably −20 to 20° C. The precipitated product is collected, for example by centrifugation at low temperatures of in general from 0 to 20° C., preferably 0° C., re-suspended, preferably with the solvent or solvent mixture, in one embodiment with the alcohol, which was used for precipitation, at temperatures usually of from −60 to 20° C., preferably −20 to 20° C., and incubated usually at the same temperature for in general 0.5 to 20 h, preferably 1 to 3 h. The obtained product is usually worked up further by centrifugation, dissolving of the product in water, dialysing in water for usually 12 to 72 h, preferably 15 to 48 h, more preferably 15 to 25 h and lyophilizing.

In the following schemes, examples for the process for preparing functionalized polymers by reaction of a polymer of formula III and a compound of formula IV, preferably

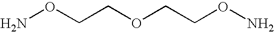

are given (schemes 1a and 2).

In scheme 1b examples for the process for preparing functionalized polymers by reaction of a polymer of formula III and a compound of formula IV, preferably

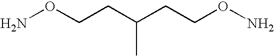

are given.

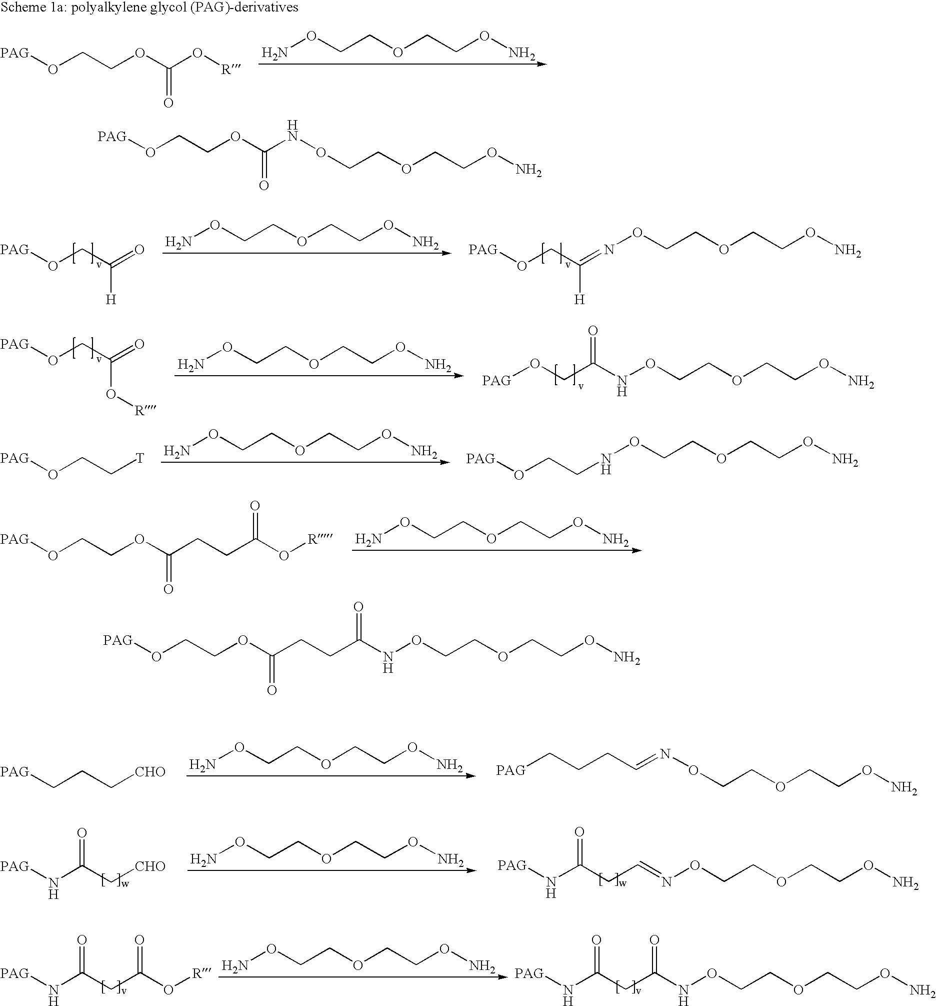

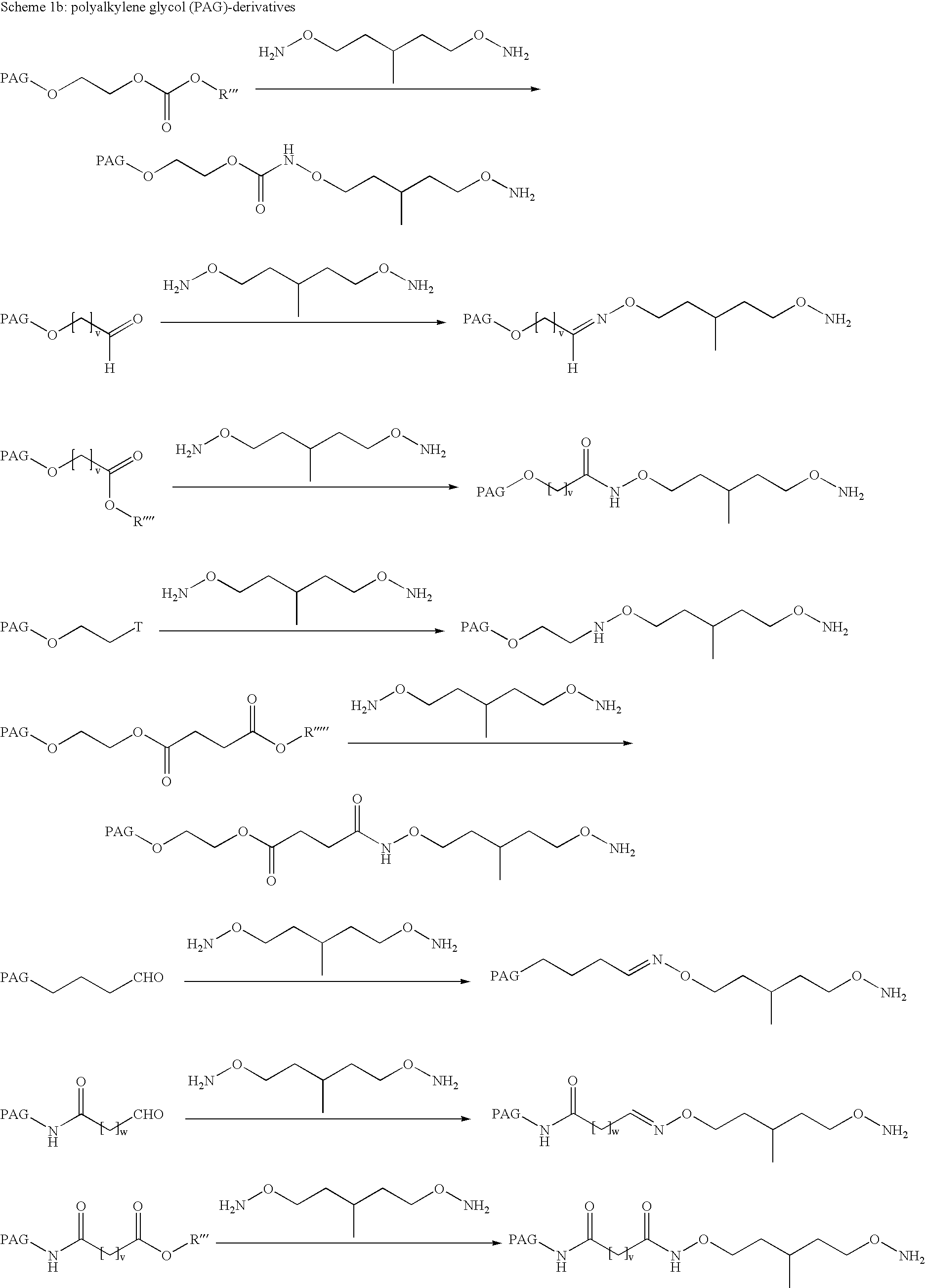

wherein OR′″, OR″″ and OR′″″ are derived from alcohols H—OR′″, H—OR″″ and H—OR′″″ said alcohols preferably being selected from the group consisting of N-hydroxy succinimides such as N-hydroxy succinimide or Sulfo-N-hydroxy succinimide, suitably substituted phenols such as p-nitrophenol, o,p-dinitrophenol, o,o′-dinitrophenol, trichlorophenol such as 2,4,6-trichlorophenol or 2,4,5-trichlorophenol, trifluorophenol such as 2,4,6-trifluorophenol or 2,4,5-trifluorophenol, pentachlorophenol, pentafluorophenol, or hydroxyazoles such as hydroxy benzotriazole. Especially preferred are N-hydroxy succinimides, with N-hydroxy succinimide and Sulfo-N-hydroxy succinimide being especially preferred, v is 1 to 10, preferably 1 to 5, more preferably 2 or 3; T is Cl, Br, I or OTf, and PEG is polyethylene glycol or a derivative thereof, preferably mPEG. PAG is polyalkylene glycol or a derivative thereof, preferably PEG, more preferably mPEG. w is 1 to 10, preferably 1 to 5, more preferably 4.

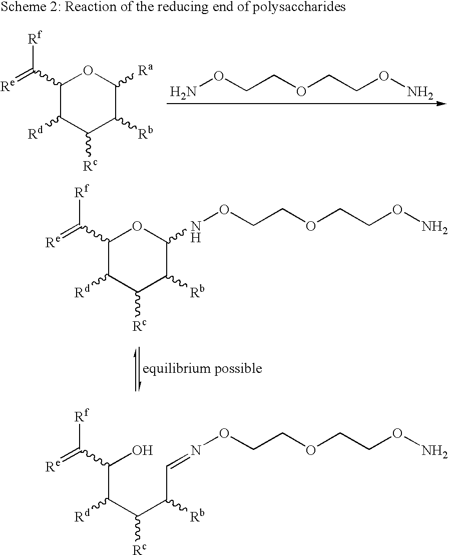

wherein R^{e} is 2H or O, R^{a} is OH, R^{b}, R^{c}, R^{d}, R^{f} are independently H, OH, O-alkyl, hydroxyalkyl, OC(O)R″″″, wherein R″″″ is alkyl, preferably O-acetyl, OPO₃H₂, OSO₃H, ONO₂ or O-polysaccharide; e.g. for dextran and derivatives thereof. R^{f} is O-polysaccharide and for starch and derivatives thereof R^{d} is O-polysaccharide.

A further embodiment of the present invention relates to polymers as obtainable by a process as mentioned before. Preferred embodiments of the process and the starting materials used are also mentioned before.

The functionalized polymer obtainable by the process mentioned above is preferably the functionalized polymer of formula I.

The functionalized polymers of the present invention are suitable as starting materials for the preparation of a conjugate of the functionalized polymer and a protein. In the conjugate a polymer selected from soluble linear or branched homopolymers or random copolymers and derivatives thereof selected from the group consisting of alkylene glycol homopolymers, preferably ethylene glycol homopolymers (PEG), propylene glycol homopolymers alkylene glycol copolymers, preferably propylene oxide/ethylene oxide co-polymers, polyvinyl alcohol, polyvinyl pyrrolidone, poly-1,3-dioxolane, poly-1,3,6-trioxane, ethylene/maleic anhydride copolymer, polyaminoacids and polysaccharides, preferably selected from the group consisting of starch, cellulose, dextran, gum arabic, xanthan gum, inulin, ghatti gum, pectin, guar gum, gum tragacanth, agar, algin, karaya gum, carrageenan, scleroglucan, fucellaran, arabinogalacton and locust bean gum, is covalently linked by a linking group of formula (IV)

Q-[(CR¹R²)ₘO]ₙ[CR³R⁴]ₒ—ONHR⁵ (IV),

preferably by a linking group of the following formula

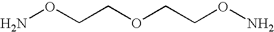

with a protein as mentioned above. Preferred polymers, linking groups and proteins are mentioned before.

In a further preferred embodiment the linking group has the formula (IV)

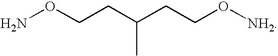

In a further embodiment the present invention relates to a process for preparing a conjugate, comprising the step of reacting a functionalized polymer of the present invention with a functionalized protein of formula V

“protein”-Z (V)

wherein Z is a group comprising a carbonyl group or a group which is suitable of forming a carbonyl group or another group which is reactable with the functionalized polymer, wherein Z is covalently linked with least one terminal group or least one centrally located group of the “protein”, preferably with an oxidized N-terminal amino acid or an oxidized carbohydrate side chain of the “protein”. Preferred functionalized polymers and proteins are mentioned before.

According to one embodiment of the present invention, the functional group Z of the protein is a group which is reactable with the polymer functionalized by a linking group having the reactable end group —O—NHR⁵ (preferably the functionalized polymer of formula I), wherein R⁵ is hydrogen, allyl or aryl, preferably hydrogen. Preferred groups Z are therefore aldehyde groups, keto groups, carboxy groups, and activated carboxy groups, for example ester groups, and lactone groups. Further suitable groups Z are halide or pseudo halide groups and the like, for example Cl, Br, I, or OTf Preferably Z is an aldehyde group or a keto group. Therefore, the present invention relates to a method and conjugates as described above, wherein the functional group Z of the protein is an aldehyde group or a keto group.

While there are no general restrictions as to the location of the group Z, preferably the aldehyde or keto group within the protein, the aldehyde or keto group is, according to a preferred embodiment of the present invention, located in a carbohydrate side chain of the protein. Therefore, in the context of this embodiment, a glycosylated protein is employed.

In the context of the present invention, the term “carbohydrate side chain” refers to oligosaccharide connected covalently to an amino acid of a protein and consisting of at least two “carbohydrate moieties”. In the context of the present invention the term “carbohydrate moieties” refers to hydroxyaldehydes or hydroxyketones as well as to chemical modifications thereof (see Römpp Chemielexikon, Thieme Verlag Stuttgart, Germany, 9^{th} edition 1990, Volume 9, pages 2281-2285 and the literature cited therein). Furthermore, it also refers to derivatives of naturally occurring carbohydrate moieties like glucose, galactose, mannose, sialic acids and the like.

In an even more preferred embodiment, the aldehyde group or the keto group is a galactose residue of the carbohydrate side chain, preferably the terminal galactose residue of the carbohydrate side chain.

Oxidation of terminal carbohydrate moieties can be performed either chemically or enzymatically.

Methods for the chemical oxidation of carbohydrate moieties of polypeptides are known in the art and include the treatment with periodate (Chamow et al., 1992, J. Biol. Chem., 267, 15916-15922).

By chemically oxidizing, it is in principle possible to oxidize any carbohydrate moiety, being terminally positioned or not. However, by choosing mild reaction conditions it is possible to preferably oxidize the terminal sialic acid of a carbohydrate side chain to give the aldehyde group or the keto group.

According to a further embodiment of the present invention, said mild reaction conditions relate to reacting the protein with a suitable aqueous periodate solution, having a preferred periodate concentration in the range of from 1 to 50 mM, more preferably of from 1 to 25 mM and especially preferably of from 1 to 10 mM such as about 1 mM, and at a preferred reaction temperature of from 0 to 40° C. and especially preferably of from 0 to 21° C. such as about 0° C., and for a preferred reaction time of from 5 min to 5 b, more preferably from 10 min to 2 h and especially preferably from 10 min. to 1 h such as about 1 h. The preferred molar ratio of periodate:protein is from 1:200 to 1:1 and more preferably from 1:50 to 1:5, such as about 1:15

If e.g., EPO is chemically oxidized, it is preferred to react EPO in an aqueous medium, preferably water, with periodate solution for preferably 1 h, at a molar ratio of periodate:EPO of about 15:1, a reaction temperature of about 0° C., using an aqueous periodate solution having a concentration of about 1 mM.

Therefore, the present invention also relates to a method and a conjugate as described above, wherein, prior to the reaction of the protein and the polymer or polymer derivative, a glycosylated protein is reacted with a periodate solution to give a protein having an aldehyde group or a keto group located in the oxidized carbohydrate side chain, said reaction preferably being carried out at mild oxidation reactions. The term “mild reaction conditions” as used in this context refers to, e.g., to a 1 nM periodate solution and a reaction temperature of 0° C. in contrast to harsh conditions such as a 10 mM periodate solution and a reaction temperature of 20 to 25° C.

Alternatively, the carbohydrate side chain may be oxidized enzymatically. Enzymes for the oxidation of the individual carbohydrate side chain are known in the art, e.g. in the case of galactose the enzyme is galactose oxidase. If it is intended to oxidize terminal galactose moieties, it will be eventually necessary to remove terminal sialic acids (partially or completely) if the polypeptide has been produced in cells capable of attaching sialic acids to carbohydrate chains, e.g. in mammalian cells or in cells which have been genetically modified to be capable of attaching sialic acids to carbohydrate chains. Chemical or enzymatic methods for the removal of sialic acids are known in the art (Chaplin and Kennedy (eds.), 1996, Carbohydrate Analysis: a practical approach, especially Chapter 5 Montreuill, Glycoproteins, pages 175-177; IRL Press Practical approach series (ISBN 0-947946-44-3)).

According to another preferred embodiment of the present invention, the aldehyde group or keto group may be located at the N terminus of the protein and is accessible by suitable oxidation. Especially in the case that a hydroxy group containing amino acid is located at the N terminus of the protein at position −1, such as threonine or serine, oxidation of said N-terminal amino acid can be carried out leading to said keto group or an aldehyde group, preferably an aldehyde group. Threonine, e.g., is preferably located at the N terminus of the protein which is an expression product, e.g. produced in eukaryotic cells such as mammalian, especially human, insect or yeast cells, and which is glycosylated with mammalian or other eukaryotic carbohydrates. As method for the chemical oxidation of the suitable N-terminal amino acid, any conceivable method may be applied, with the oxidation with periodate being preferred, with mild oxidation conditions being especially preferred.

According to a further preferred embodiment of the present invention, said mild reaction conditions, with regard to the N terminal amino acid, relate to reacting the protein with a suitable aqueous periodate solution, having a preferred periodate concentration in the range of from 1 to 50 mM, more preferably of from 1 to 25 mM and especially preferably of from 1 to 10 mM such as about 1 mM, and at a preferred reaction temperature of from 0 to 40° C. and especially preferably of from 0 to 21° C. such as about 0° C., and for a preferred reaction time of from 5 min to 5 h, more preferably from 10 min to 2 h and especially preferably from 10 min. to 1 h such as about 1 h. The preferred molar ratio of periodate:protein is from 1:200 to 1:1 and more preferably from 1:50 to 1:5 such as about 15:1.

Therefore, the present invention also relates to a method and a conjugate as described above, wherein the aldehyde group or the keto group is located in a carbohydrate side chain of the protein and/or at the N-terminal group of the protein.

In a preferred embodiment of the process for preparing the conjugate of the functionalized polymer and the functionalized protein of formula V to an aqueous solution of the functionalized protein, preferably in a sodium actetate buffer at a pH of 5.0 to 5.5, was added an aqueous solution of the functionalized polymer, preferably in a sodium actetate buffer at a pH of 5.0 to 5.5 usually at a temperature of from 0 to 40° C., preferably of from 0 to 25° C., more preferably of from 15 to 25° C. The solution is then usually incubated in general at the temperature mentioned before. The incubation is usually carried out for 3 to 72 h, preferably 8 to 48 h, more preferably 15 to 25 h. The steps for work up and isolation of the conjugate are known by a person skilled in the art. The conjugate may be subjected to a further treatment such as an after-treatment like dialysis, centrifugal filtration or a pressure filtration, ion exchange chromatography, reversed phase chromatography, HPLC, MPLC, gel filtration and/or lyophilization.

The molar ratio of the functionalized polymer of formula III and the functionalized protein of formula V is usually of from 1-50:1, preferably of from 1-30:1, more preferably of from 1-20:1, even more preferably of from 1-15:1, and most preferably of from 1-5:1, when the functionalized polymer of formula III is precipitated by adding an alcohol, preferably 2-propanol or ethanol.

The molar ratio of the functionalized polymer of formula III and the functionalized protein of formula V is usually of from 1-200:1, preferably of from 1-100:1, more preferably of from 1-50:1, when the functionalized polymer of formula III is precipitated by adding a solvent mixture of ethanol and acetone.

It is further possible to prepare the conjugate of the present invention in a two step process starting from a fuctionalized polymer of formula III.

In a further embodiment the present invention therefore relates to a process for preparing a conjugate, comprising the steps
a) reacting a polymer of formula III

“polymer”-(CR⁸R⁹)ₚ—Y (III)

with a compound of formula IV

Q-[(CR¹R²)ₘO]ₙ[CR³R⁴]ₒ—ONHR⁵ (IV),

wherein the symbols have the following meanings
“polymer” soluble linear or branched homopolymers or random copolymers and derivatives thereof selected from the group consisting of allylene glycol homopolymers, preferably ethylene glycol homopolymers (PEG), propylene glycol homopolymers, alkylene glycol copolymers, preferably propylene oxide/ethylene oxide co-polymers, polyvinyl alcohol, polyvinyl pyrrolidone, poly-1,3-dioxolane, poly-1,3,6-trioxane, ethylene/maleic anhydride copolymer, polyaminoacids and polysaccharides, preferably selected from the group consisting of starch, cellulose, dextran, gum arabic, xanthan gum, inulin, ghatti gum, pectin, guar gum, gum tragacanth, agar, algin, karaya gum, carrageenan, scleroglucan, fucellaran, arabinogalacton and locust bean gum;
R¹, R², R³, R⁴, R⁵ hydrogen, allyl, aryl, preferably hydrogen;
m 2 to 4, wherein the residues R¹ and R² may be the same or different in the m groups CR¹R²;
n 0 to 20, preferably 0 to 10, more preferably 1 to 5, most preferably 1 or 2, and even more preferably 1;
0 to 20, preferably 0 to 10, more preferably 0 or 2, wherein in the case of n=0, o is not 0,
   in a preferred embodiment o is 2 to 20, preferably 2 to 10, more preferably 2,
   wherein the residues R³ and R⁴ may be the same or different in the o groups CR³R⁴;
Y and Q functional groups, which are suitable to react together to give one of the following linking groups —O—, —S—, NR⁶—, —OC(O)—, —C(O)O—, —C(G)N(R¹⁰)O—, —N(R¹¹)O—,

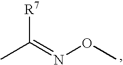

preferably

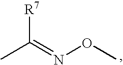

—N(R¹¹)O—, —C(G)N(R¹⁰)O—,
wherein one or more groups —(CR⁸R⁹)— may be replaced by W, whereby a chemically reasonable group is formed;
w O, NR¹², C(G), preferably O, C(G);
G S, O, NR¹⁴, preferably O;
R⁶, R⁷, R⁸, R⁹, R¹⁰, R¹¹, R¹², R¹⁴ hydrogen, alkyl, aryl, preferably hydrogen;
p 0 to 20, preferably 0 to 10, more preferably 0 to 5, most preferably 0 to 4, even more preferably 0 in the case of polysaccharides and derivatives thereof, and 1 to 4 in the case of polyalkylene glycols and derivatives thereof, wherein the residues R⁸ and R⁹ may be the same or different in the p groups CR⁸R⁹;
wherein the group

—(CR⁸R⁹)ₚ—Y
is covalently linked with least one terminal group and/or least one centrally located group of the “polymer”, wherein a functionalized polymer is obtained, and
b) reacting the functionalized polymer obtained in step a) with a functionalized protein of formula V

“protein”-Z (V)

wherein Z is a group comprising a carbonyl group or a group which is suitable of forming a carbonyl group or another group which is reactable with the functionalized polymer, wherein Z is covalently linked with least one terminal group and/or least one centrally located group of the “protein”, preferably with an oxidized N-terminal amino acid or an oxidized carbohydrate side chain of the “protein”. Preferred polymers of formula III, compounds of formula IV and functionalized proteins of formula V are the same as mentioned before.

The reaction conditions of steps a) and b) are the same as mentioned for the preparation of the functionalized polymer, preferably the polymer of formula I (step a)) and for the preparation of the conjugate starting from the functionalized polymer (step b)). The advantage of the two step process is that the isolation step of the functionalized polymer obtained in step a) may be omitted.

The conjugates of the present invention themselves or a pharmaceutical composition comprising the conjugates are useful in a method for a treatment of the human or animal body.

In a further embodiment the present invention relates to the conjugate of the present invention, or the conjugate, obtainable by a method of the present invention, for use in a method for the treatment of the human or animal body.

The pharmaceutical compositions comprising a therapeutically effective amount of the conjugate of the present invention as well as pharmaceutically acceptable diluent, adjuvant or carrier. The pharmaceutically compositions further optionally comprising further therapeutical or galenic components and adjuvants. Suitable adjuvants are for example diluents, buffer systems, binders, surface active components, thickening agents, lubricants and antidegradants (enclosing antioxidants).

A therapeutically effective amount is the amount which is sufficient to achieve a positive effect in a singular or repeatedly treatment within the scope of a treatment for facilitation, healing or prevention of a disease.

A pharmaceutically acceptable diluent is a diluent which is compatible with both, the conjugate of the present invention and the human or animal body.

The form of the pharmaceutical composition is depending on the desired or suitable way of application. The preferred application is the parenteralic application. Suitable parenteralic applications are known in the art. Further possible applications are the intranasalic, intrachealic, or topic application. The pharmaceutical compositions may be presented in form of a dose unit and are prepared by a process known in the art.

A pharmaceutical composition comprising in a therapeutically effective amount the conjugate of the present invention, or the conjugate, obtainable by a method of the present invention.

Preferably the pharmaceutical composition further comprising at least one pharmaceutically acceptable diluent, adjuvant, or carrier. Suitable diluents, adjuvants, or carriers as well as further suitable ingredients are known by a person skilled in the art.

The following examples additionally describe the present invention.

### EXAMPLES

### A) Synthesis of Hydroxylamino-PEG-Derivatives

### Aa) Synthesis of Hydroxylamino-PEG20 in Aqueous Solution

200 mg of mPEG-Butyraldehyde (mPEG-ButyrALD, MW20,000, Nektar, Huntsville, Ala., USA) were dissolved in 2 mL 0.1M sodium acetate buffer, pH 5.2 and 1 mmol O-[2-(2-aminooxy-ethoxy)-ethyl]-hydroxylamine (synthesized according to D. Boturyn et al., Tetrahedron 53 (1997) 5485-92, pp. 5489-90) were added. After shaking for 19 h at 22° C., the reaction mixture was added to 45 mL 2-propanol at −20° C. and incubated at −20° C. for 4 h. The precipitated product was collected by centrifugation at 0° C., washed with 15 mL 2-propanol at −20° C. and incubated at −20° C. for 1 h. After centrifugation, the product was dissolved in 15 mL water, dialysed for 21 h against water (SnakeSkin dialysis tubing, 3.5 kD cut off, Perbio Sciences Deutschland GmbH, Bonn, D) and lyophilized.

### Ab) Synthesis of Hydroxylamino-PEG20 in Organic Solvents

200 mg of mPEG-Butyraldehyde (mPEG-ButyrALD, MW20,000, Nektar, Huntsville, Ala., USA) were dissolved in 2 mL dichloromethane and 1 mmol O-[2-(2-aminooxy-ethoxy)-ethyl]-hydroxylamine (synthesized according to D. Boturyn et al., Tetrahedron 53 (1997) 5485-92, pp. 5489-90) were added. After shaking for 19 h at 22° C., the reaction mixture was added to 45 mL 2-propanol at −20° C. and incubated at −20° C. for 4 h. The precipitated product was collected by centrifugation at 0° C., washed with 15 mL 2-propanol at −20° C. and incubated at −20° C. for 1 h. After centrifugation, the product was dissolved in 15 mL water, dialysed for 21 h against water (SnakeSkin dialysis tubing, 3.5 kD cut off, Perbio Sciences Deutschland GmbH, Bonn, D) and lyophilized.

### B) Synthesis of a Hydroxylamino-Dextran-Derivate

### Synthesis of Hydroxylamino-Dextran17 in Aqueous Solution

500 mg of Dextran (Dextran from Leuconostoc ssp., Mᵣ ˜15000-20000D, Fluka, Sigma-Aldrich Chemie GmbH, Taufkirchen, D) were dissolved in 5 mL 0.1M sodium acetate buffer, pH 5.2 and 1 mmol O-[2-(2-aminooxy-ethoxy)-ethyl]-hydroxylamine (synthesized according to D. Boturyn et al., Tetrahedron 53 (1997) 5485-92, pp. 5489-90) were added. After shaking for 19 h at 22° C., the reaction mixture was added to 45 mL 2-propanol at −20° C. and incubated at −20° C. for 4 h. The precipitated product was collected by centrifugation at 0° C., washed with 15 mL 2-propanol at −20° C. and incubated at −20° C. for 1 h. After centrifugation, the product was dissolved in 15 mL water, dialysed for 21 h against water (SnakeSkin dialysis tubing, 3.5 kD cut off, Perbio Sciences Deutschland GmbH, Bonn, D) and lyophilized.

### C) Synthesis of the EPO Conjugates

### C1) Periodate Oxidation of N-Acetylaneuraminic Acid Residues by Mild Periodate Treatment of EPO

To a 2.0 mg/ml solution of EPO (recombinantly produced EPO having amino acid sequence of human EPO and similar or essentially the same characteristics as the commercially available (Epoietin alpha: Erypo, ORTHO BIOTECH, Jansen-Cilag or Epoietin beta: NeoRecormon, Roche; cf. EP 0 148 605, EP 0 205 564, EP 0 411 678) of total 20 ml kept at 0° C. were added 2.2 ml of an ice-cold solution of 10 mM sodium meta-periodate resulting in a final concentration of 1 mM sodium meta-perjodate. The mixture was incubated at 0° C. for 1 hour in an ice-bath in the dark and the reaction was terminated by addition of 40 μl of glycerol and incubated for further 5 minutes.

C2) Buffer Exchange of Periodate Oxidised EPO for Subsequent Derivatisation with a Hydroxylamino Functionalized Hydroxyethyl Starch Derivative

Buffer exchange was performed using a 20 ml Vivaspin 20 concentrator (Vivaspin AG, Hannover, Germany) with a polyethersulfone (PES) membrane. The concentrator unit was washed by addition of 5 ml of 0.1 M Na-acetate buffer pH 5.5 and centrifugation of the concentrator unit at 4000 rpm at 6° C. in a Megafuge 1.0R (Kendro Laboratory Equipment, Osterode, Germany). Subsequently, 20 ml of the perjodate oxidised EPO solution according to Example 1 was added to the concentrator unit and was centrifuged at 4000 rpm for 25 min until a 5-fold concentration was achieved. 15 ml of 0.1 M Na-acetate buffer pH 5.5 was added to the concentrate which was centrifuged as described above. The centrifugation cycle was repeated 3 times, the final concentrate was removed and transferred into a 50 ml sterile plastic tube, after washing of the concentrator unit 2 times with each 1 ml of Na-acetate buffer pH 5.5; the volume of the EPO was adjusted with Na-acetate buffer pH 5.5 to 26.7 ml and protein concentration of the final oxidised EPO solution was determined by measuring the absorbance at 280 nm using the specific absorbance value of 7.43 as described in the European Pharmacopeia (Erythropoietin Concentrated Solution, 4th Edition, 2002, pages 1123-1128). A value of 1.378 mg/ml was determined for the final periodate oxidised EPO solution (36.8 mg EPO, corresponding to ≈90% final yield).

C3) Synthesis of the EPO-Conjugates of mPEG Aldehyde-Reactive Polymer and Dextran Aldehyde-Reactive Polymer

To 13.3 μL of a solution of oxidized EPO in 0.1 M sodium acetate buffer, pH 5.5 (oxidation according to example C1), 6.67 μL of a solution of the aldehyde-reactive polymer in 0.1 M sodium acetate buffer, pH 5.5 were added and the solution was incubated for 22 h at 22° C. The following concentrations were employed:

100 mg/mL for aldehyde-reactive polymer prepared according to example Aa) and Ab) 87.5 mg/mL for dextran aldehyde-reactive polymer prepared according to example B)

In Examples C1) to C3), a successful conjugation is indicated by the migration of the protein bands to higher molecular weights in the SDS page analysis according to FIG. 2. The increased band-width is due to the molecular weight distribution of the dextran and PEG-derivatives used and the number of HES derivatives linked to the protein. In contrast to the hydroxylamino-dextran derivative, the corresponding PEG derivative migrates into the gel as well and is also stained, therefore complicating the visualisation of the protein bands.

### C4) Synthesis of Conjugates of Hydroxyethyl Starch and EPO

### C4.1) Synthesis of Hydroxylamino Functionalized HES Derivatives

O-[2-(2-aminooxy-ethoxy)-ethyl]-hydroxylamine was synthesized as described in Boturyn et al. Tetrahedron 53 (1997) p. 5485-5492 in 2 steps from commercially available materials.

### C4.1) (a) Synthesis of Hydroxylamino-HES 10/0.4

2 g of HES10/0.4 (MW=10000 D, DS=0.4, Supramol Parenteral Colloids GmbH, Rosbach-Rodheim, D) were dissolved in 17 mL 0.1M sodium acetate buffer, pH 5.2 and 20 mmol O-[2-(2-aminooxy-ethoxy)-ethyl]-hydroxylamine were added. After shaking for 19 h at 22° C., the reaction mixture was added to 100 mL of an ice-cold 1:1 mixture of acetone and ethanol (v/v). The precipitated product was collected by centrifugation at 4° C., re-dissolved in 50 mL water, dialysed for 21 h against water (SnakeSkin dialysis tubing, 3.5 kD cut off, Perbio Sciences Deutschland GmbH, Bonn, D) and lyophilized.

### C4.1) (b) Synthesis of Hydroxylamino-HES 10/0.7

2 g of HES10/0.7 (MW=10000 D, DS=0.7, Supramol Parenteral Colloids GmbH, Rosbach-Rodheim, D) were dissolved in 18 mL 0.1M sodium acetate buffer, pH 5.2 and 20 mmol O-[2-(2-aminooxy-ethoxy)-ethyl]-hydroxylamine were added. After shaking for 19 h at 22° C., the reaction mixture was added to 100 mL of an ice-cold 1:1 mixture of acetone and ethanol (v/v). The precipitated product was collected by centrifugation at 4° C., re-dissolved in 50 mL water, dialysed for 21 h against water (SnakeSkin dialysis tubing, 3.5 kD cut off, Perbio Sciences Deutschland GmbH, Bonn, D) and lyophilized.

### C4.1) (c) Synthesis of Hydroxylamino-HES 50/0.4

2 g of HES50/0.4 (MW=50000 D, DS=0.4, Supramol Parenteral Colloids GmbH, Rosbach-Rodheim, D) were dissolved in 20 mL 0.1M sodium acetate buffer, pH 5.2 and 4 mmol 0-[2-(2-aminooxy-ethoxy)-ethyl]-hydroxylamine were added. After shaking for 19 h at 22° C., the reaction mixture was added to 100 mL of an ice-cold 1:1 mixture of acetone and ethanol (v/v). The precipitated product was collected by centrifugation at 4° C., re-dissolved in 50 mL water, dialysed for 21 h against water (SnakeSkin dialysis tubing, 3.5 kD cut off, Perbio Sciences Deutschland GmbH, Bonn, D) and lyophilized.

### C4.1) (d) Synthesis of Hydroxylamino-HES 50/0.7

2 g of HES50/0.7 (MW=50000 D, DS=0.7, Supramol Parenteral Colloids GmbH, Rosbach-Rodheim, D) were dissolved in 20 mL 0.1M sodium acetate buffer, pH 5.2 and 4 mmol O-[2-(2-aminooxy-ethoxy)-ethyl]-hydroxylamine were added. After shaking for 17.5 h at 22° C., the reaction mixture was added to 70 mL of an ice-cold 1:1 mixture of acetone and ethanol (v/v). The precipitated product was collected by centrifugation at 0° C., washed with 30 mL of an ice-cold 1:1 mixture of acetone and ethanol (v/v), re-dissolved in 50 mL water, dialysed for 19.5 h against water (SnakeSkin dialysis tubing, 3.5 kD cut off, Perbio Sciences Deutschland GmbH, Bonn, D) and lyophilized.

### C4.2) Synthesis of HES-EPO Conjugates

In Examples C4.2) (a) to C4.2) (d), a successful conjugation is indicated by the migration of the protein bands to higher molecular weights in the SDS page analysis according to FIG. 1. The increased band-with is due to the molecular weight distribution of the HES derivatives used and the number of HES derivatives linked to the protein.

C4.2) (a) Synthesis with of Hydroxylamino-HES10/0.4 According to Example C4.1) (a)

To 3.63 mL of a solution of oxidized EPO in 0.1 M sodium acetate buffer, pH 5.5 (according to Example C2); 1.378 mg/ml), 83 mg of hydroxylaminoHES10/0.4, produced according to example C4.1) (a), were added and the solution was shaken for 16.5 h at 22° C.

C4.2) (b) Synthesis with of Hydroxylamino-HES 10/0.7 According to Example C4.1) (b)

To 3.63 mL of a solution of oxidized EPO in 0.1 M sodium acetate buffer, pH 5.5 (according to Example C2); 1.378 mg/ml), 83 mg of hydroxylaminoHES10/0.7, produced according to example C4.1) (b), were added and the solution was shaken for 16.5 h at 22° C.

C4.2) (c) Synthesis with of Hydroxylamino-HES 50/0.4 According to Example C4.1) (c)

To 3.63 mL of a solution of oxidized EPO in 0.1 M sodium acetate buffer, pH 5.5 (according to Example C2); 1.378 mg/ml), 416 mg of hydroxylaminoHES50/0.4, produced according to example C4.1) (c), were added and the solution was shaken for 16.5 h at 22° C.

C4.2) (d) Synthesis with of Hydroxylamino-HES 50/0.7 according to Example C4.1) (d)

To 3.63 mL of a solution of oxidized EPO in 0.1 M sodium acetate buffer, pH 5.5 (according to Example C2); 1.378 mg/ml), 416 mg of hydroxylaminoHES50/0.7, produced according to example C4.1) (d), were added and the solution was shaken for 16.5 h at 22° C.

D) Purification of HES-Modified EPO and Separation of Unreacted HES-Derivatives from HES-Modified EPO

Subsequent to the HES-coupling procedures according to Examples C4.2) (a) to C4.2) (d), the purification of all samples was performed at room temperature using an AKTA explorer 10 system equipped with a Pump P-903, Mixer M-925 with 0.6 ml chamber, Monitor pH/C-900, pump P-950 (sample pump) along with a Software Unicorn Version 3.21. Detection was at 280, 260 and 220 nm using a Monitor TV-900 with a 10 mm flow cell.

The incubation mixtures were diluted with 10 volumes of buffer A (20 mM N-morpholino propane sulfonic acid adjusted to pH 8.0 with NaOH) and were applied to a column containing 4 ml Q-Sepharose Fast Flow (Amersham Pharmacia Biotech) at a flow rate of 0.8 mil/min; the column was previously equilibrated with 7 column volumes (CV) of buffer A. The column was then washed with 6 CV of buffer A at a flow rate of 1.0 ml/min and elution was performed by using 2.5 CV of buffer B (0.5 M NaCl in 20 mM Na-phosphate, pH 6.5) at a flow rate of 0.6 ml/min. The column was then washed with 2.5 CV of buffer C (1.5 M NaCl in 20 mM Na-phosphate, pH 6.5) at a flow rate of 0.6 ml/min and was re-equilibrated by passing 7 CV of buffer A at flow rate of 1.0 ml/min.

Samples from incubations with (activated) hydroxylaminoHES derivatives all yielded significant absorption at 220 nm. The samples incubated with hydroxylaminoHES10/0.4 and 10/0.7, respectively, gave no detectable absorption at 280 nm, whereas the samples incubated with HydroxylaminoHES50/0.7 and 50/0.4, respectively, yielded 800 mAU×ml and 950 mAU×ml, respectively. The bound proteins were recovered in a volume of 6.5-8.0 ml almost exclusively in eluate 1, with eluate 2 containing <2% of the peak area of totally eluted peaks detected at 280 nm. The protein recovery was comparable for all EPO samples (approximately 85%).

HES-modified EPO and EPO from appropriate control incubations were subjected to buffer exchange by using 5 ml Vivaspin concentrators (10,000 MW cut-off) and centrifugation at 4000 rpm at 6° C. as described previously. Samples (1-3 mg of EPO protein) were concentrated to 0.5-0.7 ml and were diluted with phosphate buffered saline (PBS) pH 7.1 to 5 ml and subjected to 10-fold concentration by centrifugation. Each sample was subjected to the concentration and dilution cycle three times. Finally, samples were withdrawn and the concentrator units were washed with 2×0.5 ml of PBS. Samples were frozen in liquid nitrogen at protein concentrations of approximately 1.2 mg/ml.

### E) Synthesis of Crosslinking Compounds

### E1) Synthesis of O-[2-(2-aminooxy-ethoxy)-ethyl]-hydroxylamine (1)

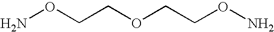

Compound (1) is synthesized according to D. Boturyn et al., Tetrahedron 53 (1997) 5485-92, pp. 5489-90, as already described in example Aa) hereinabove.

### E2) Synthesis of 1,6-Bis-aminooxy-3-methyl pentante (4)

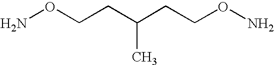

20 g (82.0 mmol) 1,5-dibromo-4-methylpentane (Acros Organics BVBA, Geel, B) were added drop-wise at room temperature to a suspension of 36.72 g (204.9 mmol) endo-N-hydroxy-5-norbornene-2,3-dicarboxamide (Lancaster Synthesis GmbH, Frankfurt/Main, D) and 36.7 g potassium carbonate (Fluka, Sigma-Aldrich Chemie GmbH, Taufkirchen, D) in 140 ml N,N-dimethylformamide (DMF) (Peptide synthesis grade, Biosolve, Valkenswaard, NL), and the reaction mixture was stirred for 90 h. The fine precipitate was removed by centrifugation, and the clear DMF supernatant was collected and concentrated in vacuo. The crude product was dried thoroughly in vacuo, dissolved in 140 ml ethanol (DAB quality, Sonnenberg, Braunschweig, D) and refluxed under nitrogen together with 16.2 ml (333.3 mmol) hydrazine hydrate (Fluka, Sigma-Aldrich Chemie GmbH, Taufkirchen, D) for 2 h. The solvent and remaining hydrazine hydrate was removed in vacuo and the crude product was suspended in 150 ml tert-butyl methyl ether (MTBE) (Acros Organics BVBA, Geel, B) and stirred for 1 h at room temperature. The precipitate was filtered of, washed with MTBE and discarded. The collected filtrates were concentrated in vacuo and the product was purified by column chromatography (3% methanol in dichloromethane, both Acros Organics BVBA, Geel, B) yielding 38% of (4) as colourless oil. δ_{II} (300 MHz; CDCl₃) 0.89 (3H, d, J 6.5), 1.37-1.44 (2H, m), 1.55-1.66 (3H, m), 3.64-371 (4H, m), 5.30 (4H, s); δ_{C} (75.5 MHz; CDCl₃) 19.7, 27.2, 35.4, 74.3.

### E3) Synthesis of 2-(2-Bromoethoxy)-3a,7a-dihydro-isoindole-1,3-dione (5)

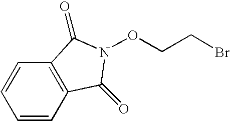

Compound (5 was synthesized as described in Bauer, L. and Suresh, K. S., J. Org. Chem. 1963, 28, p. 1604, from 16.3 g (100 mmol) N-hydroxyphthalimide (Acros Organics BVBA, Geel, B), 17.0 ml (196.9 mmol) 1,2-dibromoethane (Lancaster Synthesis GmbH, Frankfurt/Main, D) and 28 ml (197.6 mmol) triethylamine (Acros Organics BVBA, Geel, B) in 120 ml DMF (Peptide synthesis grade, Biosolve, Valkenswaard, NL). After removing of the formed precipitate by filtration, the crude product was precipitated with 800 ml water, collected by filtration and purified by column chromatography (25% tert-butyl methyl ether in petroleum ether, both Acros Organics BVBA, Geel, B) yielding 7.58 g (29%) of (5) as an off white solid. HER (400 MHz; CDCl₃) 3.63 (3H, t, J 6.9), 4.46 (3H, t, J 6.9), 7.75 (2H, dd, J 3.1 and 5.5), 7.83 (2H, dd, J 3.1 and 5.5).

### E4) Synthesis of 2-(6-Bromo-hexyloxy)-3a,7a-dihydro-isoindole-1,3-dione (6)

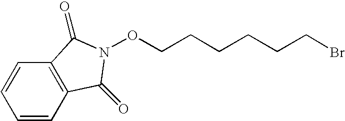

Compound (6) was synthesized as described in Bauer, L. and Suresh, K. S., J. Org. Chem. 1963, 28, p. 1604, from 16.3 g (100 mmol) N-hydroxyphthalimide (Acros Organics BVBA, Geel, B), 30.8 ml (200 mmol) 1,6-dibromohexane (Aldrich, Sigma-Aldrich Chemie GmbH, Taufkirchen, D) and 28 ml (197.6 mmol) triethylamine (Acros Organics BVBA, Geel, B) in 120 ml DMF (Peptide synthesis grade, Biosolve, Valkenswaard, NL). Purification by column chromatography (10% tert-butyl methyl ether in petroleum ether, both Acros Organics BVBA, Geel, B) yielded 12.28 g (38%) of (6) as an off white solid. δ_{H} (400 MHz; CDCl₃) 1.48-1.60 (4H, m), 1.77-1.84 (2H, m), 1.87-1.93 (2H, m), 3.42 (2H, t, J 6.8), 4.20 (2H, t, J 6.5), 7.71-7.76 (2H, m), 7.81-7.85 (2H, m); δ_{C} (100.6 MHz; CDCl₃) 24.9, 27.9, 28.0, 32.7, 33.7, 78.4, 123.5, 129.1, 134.5, 163.7.

### F) Preparation of reactive Polymer-Derivatives of the Hydroxylamine Crosslinker

### F1) Synthesis of Hydroxylamino-Dextran 17.5 (A) from Crosslinker (4) and Dextran 15-20 kD

204 mg of Dextran 15-20 kD (MW=15000-20000 D, Fluka, Sigma-Aldrich Chemie, Taufkirchen, D) were dissolved in 2 mL 0.1M sodium acetate buffer, pH 5.2 and 0.57 mmol of (4) were added. After shaking for 17 h at 22° C., the reaction mixture was added to 40 mL of ice-cold 2-propanol and incubated at −20° C. for 1 h. The precipitated product was collected by centrifugation at 4° C., suspended in 15 mL 2-propanol and again collected by filtration. The crude product was dissolved in 15 ml water, dialysed for 43.5 h against water (SnakeSkin dialysis tubing, 3.5 kD cut off, Perbio Sciences Deutschland GmbH, Bonn, D) and lyophilized. The yield of isolated product was 82%.

### F2) Synthesis of Hydroxylamino-HES10/0.4 (B) from Crosslinker(4) and HES10/0.4

403 mg of HES10/0.4 (MW=10000 D, DS=0.4, Supramol Parenteral Colloids GmbH, Rosbach-Rodheim, D) were dissolved in 4 mL 0.1M sodium acetate buffer, pH 5.2 and 4 mmol of (4) were added. After shaking for 17 h at 22° C., the reaction mixture was added to 40 mL of ice-cold 2-propanol and incubated at −20° C. for 1 h. The precipitated product was collected by centrifugation at 4° C., suspended in 15 mL

2-propanol and again collected by filtration. The crude product was dissolved in 15 ml water, dialysed for 46 h against water (SnakeSkin dialysis tubing, 3.5 kD cut off, Perbio Sciences Deutschland GmbH, Bonn, D) and lyophilized. The yield of isolated product was 52%.

### F3) Synthesis of Hydroxylamino-PEG10 (C) from Crosslinker (1) and mPEG-NHS Ester 10 kD

607 mg of α-methoxy-PEG-ω-succinimidyl ester (MW 10000 D, Rapp Polymere, Tübingen, D) were dissolved in 6 mL dichloromethane (Acros Organics BVBA, Geel, B) and 3.2 mmol of 1 were added. After shaking for 17 h at 22° C., the reaction mixture was added to 40 mL of ice-cold 2-propanol and incubated at −20° C. for 1 h. The precipitated product was collected by centrifugation at 4° C., suspended in 15 mL 2-propanol and again collected by filtration. The crude product was dissolved in 15 ml water, dialysed for 43.5 h against water (SnakeSkin dialysis tubing, 3.5 kD cut off, Perbio Sciences Deutschland GmbH, Bonn, D) and lyophilized. The yield of isolated product was 82%.

200 mg of this product was dissolved in 2 ml dichloromethane (Acros Organics BVBA, Geel, B) and 0.2 mmol morpholine (Acros Organics BVBA, Geel, B) was added. After shaking for 17 h at 22° C., the reaction mixture was added to 40 mL tert-butyl methyl ether (Acros Organics BVBA, Geel, B) and incubated at −20° C. for 1 h. The precipitated product was collected by centrifugation at 4° C., suspended in 15 mL tert-butyl methyl ether and again collected by filtration. The crude product was dissolved in 10 ml water, dialysed for 45 h against water (SnakeSkin dialysis tubing, 3.5 kD cut off, Perbio Sciences Deutschland GmbH, Bonn, D) and lyophilized. The yield of isolated product was 79%.

### F4) Synthesis of Hydroxylamino-PEG10 (D) from Crosslinker (4) and mPEG-NHS Ester 10 kD

206 mg of α-methoxy-PEG-ω-succinimidyl ester (MW=10000 D, Rapp Polymere, Tübingen, D) were dissolved in 6 mL dichloromethane (Acros Organics BVBA, Geel, B) and 1 mmol of (4) was added. After shaking for 17 h at 22° C., the reaction mixture was added to 40 mL of ice-cold 2-propanol and incubated at −20° C. for 1 h. The precipitated product was collected by centrifugation at 4° C., suspended in 15 mL 2-propanol and again collected by filtration. The crude product was dissolved in 15 ml water, dialysed for 43.5 h against water (SnakeSkin dialysis tubing, 3.5 kD cut off, Perbio Sciences Deutschland GmbH, Bonn, D) and lyophilized. The yield of isolated product was 86%.

100 mg of this product was dissolved in 2 ml dichloromethane (Acros Organics BVBA, Geel, B) and 0.2 mmol morpholine (Acros Organics BVBA, Geel, B) was added. After shaking for 17 h at 22° C., the reaction mixture was added to 40 mL tert-butyl methyl ether (Acros Organics BVBA, Geel, B) and incubated at −20° C. for 1 h. The precipitated product was collected by centrifugation at 4° C., suspended in 15 mL tert-butyl methyl ether and again collected by centrifugation. The crude product was dissolved in 10 ml water, dialysed for 45 h against water (SnakeSkin dialysis tubing, 3.5 kD cut off, Perbio Sciences Deutschland GmbH, Bonn, D) and lyophilized. The yield of isolated product was 61%.

### F5) Synthesis of HydroxylaminoHES10/0.4 ($) from Crosslinker (4) and Oxidized HES10/0.4

400 mg of oxidized HES10/0.4 (MW=10000 D, DS=0.4, Supramol Parenteral Colloids GmbH, Rosbach-Rodheim, D) were heated at 80° C. in vacuo for 17 h and dissolved in 4 mL dry DMSO (Fluka, Sigma-Aldrich Chemie GmbH, Taufkirchen, D). To the solution 4 mmol (4) were added. After incubation for 5 d at 65° C., the reaction mixture was added to 35 mL of ice-cold 2-propanol and was incubated at −20° C. for 1 h. The fine precipitated product was collected by centrifugation at 4° C. for 9 h, re-dissolved in 10 mL water, dialysed for 47 h against water (SnakeSkin dialysis tubing, 3.5 kD) cut off, Perbio Sciences Deutschland GmbH, Bonn, D) and lyophilized.

### F6) Synthesis of Hydroxylamino-PEG10 (F) from Crosslinker (1) and mPEG-Aldehyde 10 kD

604 mg of α-methoxy-PEG-ω-aldehyde (MW=10000 D, Rapp Polymere, Tübingen, D) were dissolved in 6 mL 4 mL 0.1M sodium acetate buffer, pH 5.2 and 2.2 mmol of (1) were added. After shaking for 17 h at 22° C., the reaction mixture was concentrated it vacuo, redissolved in 6 ml dichloromethane (Acros Organics BVBA, Geel, B), added to 40 mL of ice-cold 2-propanol and incubated at −20° C. for 1 h. The precipitated product was collected by centrifugation at 4° C., suspended in 15 mL 2-propanol and again collected by centrifugation. The crude product was dissolved in 15 ml water, dialysed for 42 h against water (SnakeSkin dialysis tubing, 3.5 kD cut off, Perbio Sciences Deutschland GmbH, Bonn, D) and lyophilized. The yield of isolated product was 87%.

### F7) Synthesis of Hydroxylamino-PEG10 (G) from Crosslinker (4) and mPEG-Aldehyde 10 kD

200 mg of α-methoxy-PEG-ω-aldehyde (MW=10000 D, Rapp Polymere, Tübingen, D) were dissolved in 6 mL 4 mL 0.1M sodium acetate buffer, pH 5.2 and 1 mmol of (4) was added. After shaking for 17 h at 22° C., the reaction mixture was added to 40 mL of ice-cold 2-propanol and incubated at −20° C. for 1 h. The precipitated product was collected by centrifugation at 4° C., suspended in 15 mL 2-propanol and again collected by centrifugation. The crude product was dissolved in 15 ml water, dialysed for 43.5 h against water (SnakeSkin dialysis tubing, 3.5 kD cut off, Perbio Sciences Deutschland GmbH, Bonn, D) and lyophilized. The yield of isolated product was 60%.

### F8) Synthesis of Hydroxylamino-PEG10 (H) from Crosslinker (6) and mPEG-thiol 10 kD

300 mg of α-methoxy-PEG-ω-thiol (MW=10000 D, Rapp Polymere, Tubingen, D) were dissolved in 3 mL N,N-dimethylformamide (DMF) (Peptide synthesis grade, Biosolve, Valkenswaard, NL) and oxygen was removed with a stream of nitrogen. This solution was added under nitrogen to 0.4 mmol of (6) and 0.44 mmol caesium carbonate (Fluka, Sigma-Aldrich Chemie GmbH, Taufkirchen, D). After stirring for 2.5 h at 22° C., the reaction mixture was added to 40 mL tert-butyl methyl ether (Acros Organics BVBA, Geel, B) and incubated at −20° C. for 1 h. The precipitated product was collected by centrifugation at 4° C., suspended in 15 mL tert-butyl methyl ether, again collected by centrifugation and dried in vacuo. The yield of isolated product was not determined.

150 mg of this crude product was dissolved in 1.5 ml dichloromethane (Acros Organics BVBA, Geel, B) and 0.15 mmol hydrazine hydrate (Fluka, Sigma-Aldrich. Chemie GmbH, Taufkirchen, D) were added. After shaking for 17 h at 22° C., the reaction mixture was added to 40 mL tert-butyl methyl ether and incubated at −20° C. for 1 h. The precipitated product was collected by centrifugation at 4° C., suspended in 15 mL tert-butyl methyl ether and again collected by centrifugation. The crude product was dissolved in 10 ml water, dialysed for 41 h against water (SnakeSkin dialysis tubing, 3.5 kD cut off, Perbio Sciences Deutschland GmbH, Bonn, D) and lyophilized. The yield of isolated product was 63%.

### F9) Synthesis of Hydroxylamino-PEG10 (I) from Crosslinker (5) and mPEG-thiol 10 kD

300 mg of α-methoxy-PEG-ω-thiol (MW=10000 D, Rapp Polymere, Tübingen, D) were dissolved in 3 mL N,N-dimethylformamide (DMF) (Peptide synthesis grade, Biosolve, Valkenswaard, NL) and oxygen was removed with a stream of nitrogen. This solution was added under nitrogen to 0.4 mmol of (5) and 0.44 mmol caesium carbonate (Fluka, Sigma-Aldrich Chemie GmbH, Taufkirchen, D). After stirring for 2.5 h at 22° C., the reaction mixture was added to 40 mL tert-butyl methyl ether (Acros Organics BVBA, Geel, B) and incubated at −20° C. for 1 h. The precipitated product was collected by centrifugation at 4° C., suspended in 15 mL tert-butyl methyl ether, again collected by centrifugation and dried in vacuo. The yield of isolated product was not determined.

150 mg of this crude product was dissolved in 1.5 ml dichloromethane (Acros Organics BVBA, Geel, B) and 0.15 mmol hydrazine hydrate (Fluka, Sigma-Aldrich Chemie GmbH, Taufkirchen, D) were added. After shaking for 17 h at 22° C., the reaction mixture was added to 40 mL tert-butyl methyl ether and incubated at −20° C. for 1 h. The precipitated product was collected by centrifugation at 4° C., suspended in 15 mL tert-butyl methyl ether and again collected by centrifugation. The crude product was dissolved in 10 ml water, dialysed for 41 h against water (SnakeSkin dialysis tubing, 3.5 kD cut off, Perbio Sciences Deutschland GmbH, Bonn, D) and lyophilized. The yield of isolated product was 70%.

### F10) Synthesis of HydroxylaminoHES18/0.5 (i) from Crosslinker (1) and Oxidized HES18/0.5

200 mg of oxidized HES18/0.5 (MW=18000 D, DS=0.5) were heated at 80° C. in vacuo for 17 h and dissolved in 2 mL dry DMSO (Fluka, Sigma-Aldrich Chemie GmbH, Taufkirchen, D). To the solution 2 mmol of (1) were added. After incubation for 5 d at 65° C., the reaction mixture was added to 20 mL of ice-cold 2-propanol and was incubated at −20° C. for 1 h. The precipitated product was collected by centrifugation at 4° C., washed with 42 ml ice-cold 2-propanol, re-dissolved in 10 mL water, dialysed for 27 h against water (SnakeSkin dialysis tubing, 3.5 kD cut off, Perbio Sciences Deutschland GmbH, Bonn; D) and lyophilized. The yield of isolated product was 72%.

### F11) Synthesis of HydroxylaminoHES10/0.4 (K) from Crosslinker (1) and HES10/0.4

0.8 g of HES10/0.4 (MW=10000 D, DS=0.4, Supramol Parenteral Colloids GmbH, Rosbach-Rodheim, D) were dissolved in 8 mL 0.1M sodium acetate buffer, pH 5.5 and 8 mmol of (1) were added. After shaking for 19 h at 22° C., the reaction mixture was added to 40 mL of 2-propanol at −20° C. The precipitated product was collected by centrifugation at 4° C., re-dissolved in 50 mL water, dialysed for 45 h against water (SnakeSkin dialysis tubing, 3.5 kD cut off, Perbio Sciences Deutschland GmbH, Born, D) and lyophilized. The isolated product yield was 73%.

### F12) Synthesis of Hydroxylamino-PEG10 (L) from Crosslinker (6) and mPEG-Alcohol 5 kD

1 g of α-methoxy-PEG-alcohol (MW=5000 D, Fluka, Sigma-Aldrich Chemie GmbH, Taufkirchen, D) and 257 mg powdered potassium hydroxide (Riedel-de Haën, Sigma-Aldrich Chemie GmbH, Taufkirchen, D) were suspended under nitrogen in 10 mL dry dimethyl sulphoxide (DMSO) (Fluka, Sigma-Aldrich Chemie GmbH, Taufkirchen, D) and the reaction mixture was heated to 70° C. 978 mg of (6) was added as a solid and the mixture was stirring for 2 h at 70° C. The resulting solution was added to 200 mL tert-butyl methyl ether (Acros Organics BVBA, Geel, B) The precipitated product gum was extracted twice with 100 mL tert-butyl methyl ether, dissolved in 40 ml water, dialysed for 47 h against water (SnakeSkin dialysis tubing, 3.5 kD cut off, Perbio Sciences Deutschland GmbH, Bonn, D) and lyophilized. The yield of isolated product was 98%.

482 mg of this crude product was dissolved in 5 ml dichloromethane (Acros Organics BVBA, Geel, B) and 1 mmol hydrazine hydrate (Fluka, Sigma-Aldrich Chemie GmbH, Taufkirchen, D) was added. After shaking for 17 h at 22° C., the reaction mixture was added to 40 mL tert-butyl methyl ether and incubated at −20° C. for 1 h. The precipitated product was collected by centrifugation at 4° C., suspended in 15 mL tert-butyl methyl ether and again collected by centrifugation. The crude product was dissolved in 10 ml water and dialysed for 41 h against water (SnakeSkin dialysis tubing, 3.5 kD cut off, Perbio Sciences Deutschland GmbH, Bonn, D). Centrifugation for 17 h removed a fine precipitate that formed during dialysis. The clear supernatant was lyophilized, yielding 67% of isolated product.

### G) Conjugation of the Reactive Polymer-Derivatives to Oxidized hEPO

G1) A solution of oxidized hEPO in 0.1 M sodium acetate buffer, pH 5.5 is obtained by a method as described in C1) and C2). The final concentration of oxidised hEPO was adjusted to 2 mg/mL
G2) Synthesis of the EPO Conjugates with the Polymers Prepared in Examples F1) to F12)

To 20 μL of a solution of oxidized hEPO in 0.1 M sodium acetate buffer, pH 5.5, (as obtained in Example G1), 20 μL of a solution of the aldehyde-reactive polymer in 0.1 M sodium acetate buffer, pH 5.5 were added and the solution was incubated for 22 h at 22° C. The concentrations according to the following Table 1 of the aldehyde-reactive polymers were employed:

TABLE 1

| | Polymer | Synthesized as | | Concentration |
|---|---|---|---|---|
| Entry | Derivative | described in | Equivalents | [mg/ml] |
| 1 | (A) | F1) | 5 | 5.83 |
| 2 | (B) | F2) | 5 | 3.33 |
| 3 | (C) | F3) | 5 | 3.33 |
| 4 | (D) | F4) | 5 | 3.33 |
| 5 | (E) | F5) | 50 | 33.3 |
| 6 | (F) | F6) | 50 | 33.3 |
| 7 | (G) | F7) | 50 | 33.3 |
| 8 | (H) | F8) | 5 | 3.33 |
| 9 | (I) | F9) | 5 | 3.33 |
| 10 | (J) | F10) | 50 | 60.0 |
| 11 | (K) | F11) | 5 | 3.33 |
| 12 | (L) | F12) | 50 | 33.3 |
| 13 | without polymer | | | |

A successful conjugation is indicated by the migration of the protein bands to higher molecular weights in the SDS page analysis according to FIG. 3-6. The increased band-width is due to the molecular weight distribution of the HES-, dextran- and PEG-derivatives used and the number of polymer derivatives linked to the protein. In contrast to the hydroxylamino-dextran or hydroxylamino-HES derivatives, the corresponding PEG derivatives migrate into the gel and are also stained, therefore complicating the visualisation of the protein bands.

### SHORT DESCRIPTION OF THE FIGURES

FIG. 1

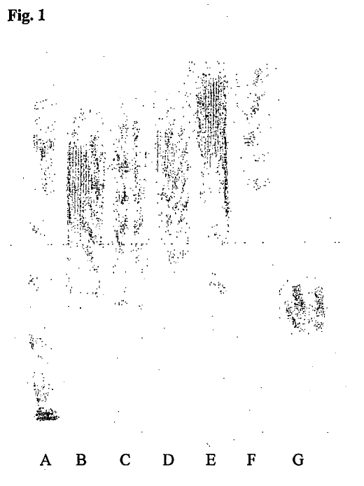

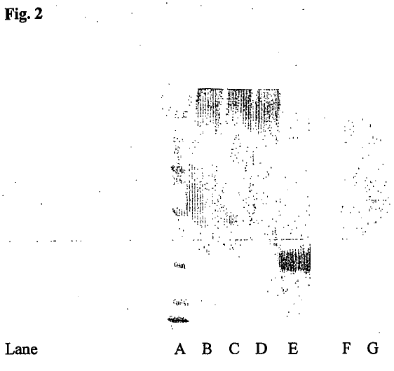

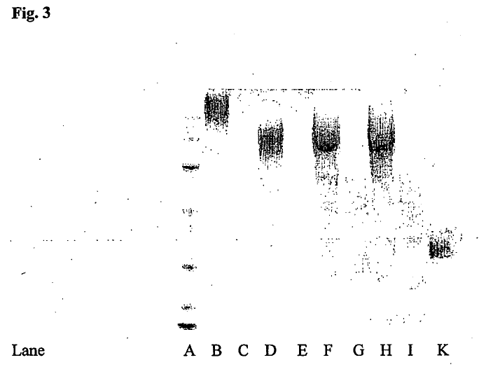

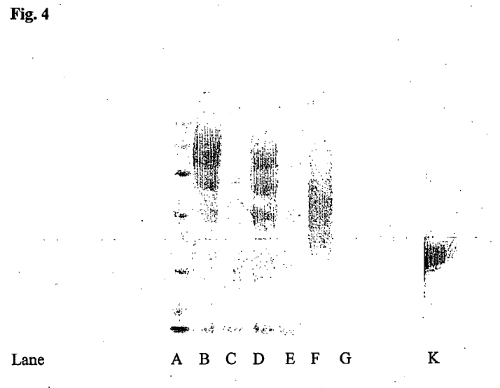

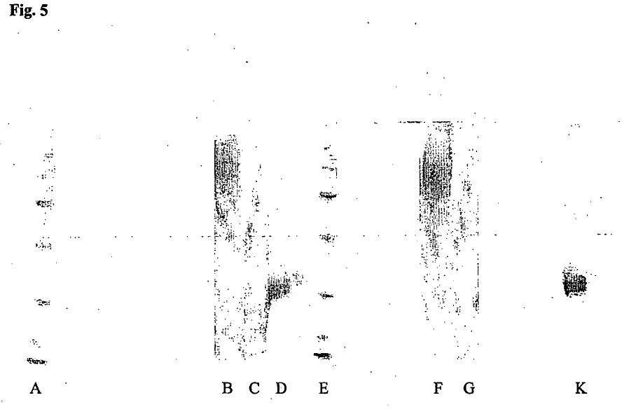

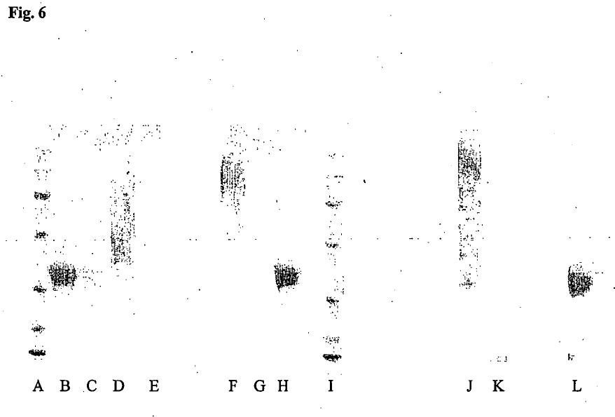

FIG. 1 shows an SDS page analysis of the HES-EPO conjugates, produced according to Example C4.2). For gel electrophoresis a XCell Sure Lock Mini Cell (Invitrogen GmbH, Karlsruhe, D) and a Consort E143 power supply (CONSORTnv, Turnhout, B) were employed. A 10% Bis-Tris gel together with a MOPS SDS running buffer at reducing conditions (both Invitrogen GmbH, Karlsruhe, D) were used according to the manufacture's instruction.
Lane A: Protein marker SeeBlue®Plus2 (Invitrogen GmbH, Karlsruhe, D) Molecular weight marker from top to bottom: 188 kD, 98 kD, 62 kD, 49 kD, 38 kD, 28 kD, 17 kD, 14 kD, 6 kD, 3 kD
Lane B: Crude reaction product Example C4.2) (a)
Lane C: Crude reaction product Example C4.2) (b)
Lane E: Crude reaction product Example C4.2) (d)
Lane F: Crude reaction product Example C4.2) (c)
Lane G: Oxidized EPO according to Example C2).

FIG. 2

FIG. 2 shows an SDS page analysis of the HES-EPO conjugates, produced according to Example C3). For gel electrophoresis a XCell Sure Lock Mini Cell (Invitrogen GmbH, Karlsruhe, D) and a Consort E143 power supply (CONSORTnv, Turnhout, B) were employed. A 10% Bis-Tris gel together with a MOPS SDS running buffer at reducing conditions (both Invitrogen GmbH, Karlsruhe, D) were used according to the manufacture's instruction.
Lane A: Protein marker Roti-Mark STANDARD (Carl Roth GmbH+Co.KG, Karlsruhe, D) Molecular weight marker from top to bottom: 200 KD, 119 KD, 66 KD, 43 KD, 29 KD, 20 KD, 14.3 KD.
Lane B: Crude product after conjugation of oxidized hEPO with polymer derivative prepared as described in Example Aa).
Lane C: Crude product after conjugation of oxidized hEPO with polymer derivative prepared as described in Example Ab).
Lane D: Crude product after conjugation of oxidized hEPO with polymer derivative prepared as described in Example B).
Lane E: Reaction control: hEPO without polymer derivative.
Lane F: Polymer derivative prepared as described in Example Aa).
Lane G: Polymer derivative prepared as described in Example Ab).

FIGS. 3-6

Each of FIGS. 3-6 shows an SDS page analysis of the HES-EPO conjugates, produced according to Example G). For gel electrophoresis a XCell Sure Lock Mini Cell (Invitrogen GmbH, Karlsruhe, D) and a Consort E143 power supply (CONSORTnv, Turnhout, B) were employed. A 10% Bis-Tris gel together with a MOPS SDS running buffer at reducing conditions (both Invitrogen GmbH, Karlsruhe, D) were used according to the manufacture's instruction.

FIG. 3
Lane A: Protein marker Roti-Mark STANDARD (Carl Roth GmbH+Co.KG, Karlsruhe, D) Molecular weight marker from top to bottom: 200 KD, 119 KD, 66 KD, 43 KD, 29 KD, 20 KD, 14.3 KD.
Lane B: Crude product after conjugation of oxidized hEPO with polymer derivative A.
Lane C: Polymer derivative A.
Lane D: Crude product after conjugation of oxidized hEPO with polymer derivative B.
Lane E: Polymer derivative B.
Lane F: Crude product after conjugation of oxidized hEPO with polymer derivative C.
Lane G: Polymer derivative C.
Lane H: Crude product after conjugation of oxidized HEPO with polymer derivative D.
Lane I: Polymer derivative D.
Lane K: Reaction control: HEPO without polymer derivative.

FIG. 4

Lane A: Protein marker Roti-Mark STANDARD (Carl Roth GmbH+Co.KG, Karlsruhe, D) Molecular weight marker from top to bottom: 200 KD, 119 KD, 66 KD, 43 KD, 29 KD, 20 KD, 14.3 KD.
Lane B: Crude product after conjugation of oxidized hEPO with polymer derivative H.
Lane C: Polymer derivative H.
Lane D: Crude product after conjugation of oxidized hEPO with polymer derivative I.
Lane E: Polymer derivative I.
Lane F: Crude products after conjugation of oxidized hEPO with polymer derivative E.
Lane G: Polymer derivative E.
Lane K: Reaction control: hEPO without polymer derivative.

FIG. 5
Lane A: Protein marker Roti-Mark STANDARD (Carl Roth GmbH+Co.KG, Karlsruhe, D) Molecular weight marker from top to bottom: 200 KD, 119 KD, 66 KD, 43 KD, 29 KD, 20 KD, 14.3 KD.
Lane B: Crude product after conjugation of oxidized hEPO with polymer derivative F.
Lane C: Polymer derivative F.
Lane D: Reaction control: hEPO without polymer derivative.
Lane E: Polymer Protein marker Roti-Mark STANDARD (Carl Roth GmbH+Co.KG, Karlsruhe, D) Molecular weight marker from top to bottom: 200 KD, 119 KD, 66 KD, 43 KD, 29 KD, 20 KD, 14.3 KD.
Lane F: Crude product after conjugation of oxidized hEPO with polymer derivative G.
Lane G: Polymer derivative G.
Lane K: Reaction control: hEPO without polymer derivative.

FIG. 6

Lane A: Protein marker Roti-Mark STANDARD (Carl Roth GmbH+Co.KG, Karlsruhe, D) Molecular weight marker from top to bottom: 200 KD, 119 KD, 66 KD, 43 KD, 29 KD, 20 KD, 14.3 KD.
Lane B: Crude product after conjugation of oxidized hEPO with polymer derivative M.
Lane C: Polymer derivative M.
Lane D: Crude product after conjugation of oxidized hEPO with polymer derivative E
Lane E: Polymer derivative J.
Lane F: Crude product after conjugation of oxidized hEPO with polymer derivative K.
Lane G: Polymer derivative K.
Lane H: Reaction control: hEPO without polymer derivative.
Lane I: Protein marker Roti-Mark STANDARD (Carl Roth GmbH+Co.KG, Karlsruhe, D) Molecular weight marker from top to bottom: 200 KD, 119 KD, 66 KD, 43 KD, 29 KD, 20 KD, 14.3 KD.
Lane J: Crude product after conjugation of oxidized hEPO with polymer derivative L.
Lane K: Polymer derivative L.
Lane L: Reaction control: hEPO without polymer derivative.

1. Functionalized polymer of formula I 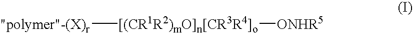
wherein the symbols have the following meanings
“polymer” soluble linear or branched homopolymers or random copolymers and derivatives thereof selected from the group consisting of alkylene glycol homopolymers, alkylene glycol copolymers, polyvinyl alcohol, polyvinyl pyrrolidone, poly-1,3-dioxolane, poly-1,3,6-trioxane, ethylene/maleic anhydride copolymer, polyaminoacids and polysaccharides;
R¹, R², R³, R⁴, R⁵ hydrogen, alkyl, aryl;
m 2 to 4, wherein the residues R¹ and R² may be the same or different in the m groups CR¹R²;
n 0 to 20;
o 0 to 20, wherein in the case of n=0, o is not 0, wherein the residues R³ and R⁴ may be the same or different in the o groups CR³R⁴;
r 0 or 1;
X —(CR⁸R⁹)ₚO—, —(CR⁸R⁹)ₚS—, —(CR⁸R⁹)ₚNR⁶—, —(CR⁸R⁹)ₚOC(O)—, —(CR⁸R⁹)ₚC(O)O—, —(CR⁸R⁹)ₚC(G)N(R¹⁰)O—, —(CR⁸R⁹)ₚN(R¹¹)O—, 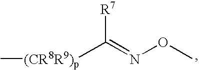
wherein one or more groups —(CR⁸R⁹)— may be replaced by W, whereby a chemically reasonable group is formed;
W O, NR¹², C(G);
G S, O, NR¹⁴;
R⁶, R⁷, R⁸, R⁹, R¹⁰, R¹¹, R¹², R¹⁴ hydrogen, alkyl, aryl,
p 0 to 20, wherein the residues R⁸ and R⁹ may be the same or different in the p groups CR⁸R⁹;
wherein the group 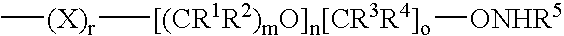
is covalently linked with at least one terminal group or at least one centrally located groups of the “polymer”.

2. Functionalized polymer as claimed in claim 1, wherein X is 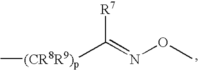
—(CR⁸R⁹)ₚN(R¹¹)O— or —(CR⁸R⁹)ₚC(G)N(R¹⁰)O—.

3. Functionalized polymer as claimed in claim 1 or 2, wherein the polymer is hydroxyalkyl starch, dextran or ethylene glycol homopolymer.

4. Functionalized polymer as claimed in any of claims 1 or 3, wherein the group
—[(CR¹R²)ₘO]ₙ[CR³R⁴]ₒ—
is —CH₂CH₂OCH₂CH₂—.

5. Conjugate of formula II 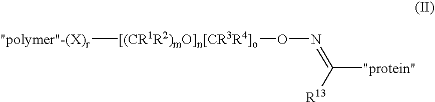
wherein the symbols have the following meanings
“polymer” soluble linear or branched homopolymers or random copolymers and derivatives thereof selected from the group consisting of alkylene glycol homopolymers, alkylene glycol copolymers, polyvinyl alcohol, polyvinyl pyrrolidone, poly-1,3-dioxolane, poly-1,3,6-trioxane, ethylene/maleic anhydride copolymer, polyaminoacids and polysaccharides;
R¹, R², R³, R⁴ hydrogen, alkyl, aryl;
m 2 to 4, wherein the residues R¹ and R² may be the same or different in the m groups CR¹R²;
n 0 to 20;
o 0 to 20, wherein in the case of n=0, o is not 0, wherein the residues R³ and R⁴ may be the same or different in the o groups CR³R⁴;
r 0 or 1;
x —(CR⁸R⁹)ₚO—, —(CR⁸R⁹)ₚS—, —(CR⁸R⁹)ₚNR⁶—, —(CR⁸R⁹)ₚOC(O)—, —(CR⁸R⁹)ₚC(O)O—, —(CR⁸R⁹)ₚC(G)N(R¹⁰)O—, —(CR⁸R⁹)ₚN(R¹¹)O—, 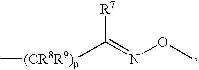
wherein one or more groups —(CR⁸R⁹)— may be replaced by W, whereby a chemically reasonable group is formed;
W O, NR¹², C(G);
G S, O, NR¹⁴;
R⁶, R⁷, R⁸, R⁹, R¹⁰, R¹¹, R¹², R¹⁴ hydrogen, alkyl, aryl, preferably hydrogen,
p 0 to 20, wherein the residues R⁸ and R⁹ may be the same or different in the p groups CR⁸R⁹;
R¹³ hydrogen, alkyl, aryl;
“protein” amino acid sequence prepared by reaction of at least 2 amino acids wherein the group 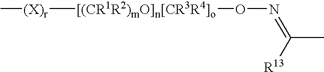
is covalently inked with at least one terminal group or at least one centrally located group of the “polymer” and the “protein”.

6. Conjugate as claimed in claim 5, wherein X is 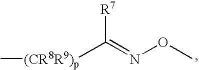
—(CR⁸R⁹)ₚN(R¹¹)O— or —(CR⁸R⁹)ₚC(6)N(R¹⁰)O—.

7. Conjugate as claimed in claim 5 or 6, wherein the polymer is hydroxyalkyl starch, dextran or ethylene glycol homopolymer.

8. Conjugate as claimed in any of claims 5 to 7, wherein the “protein” is selected from the group consisting of EPO, G-CSF, Factor VII, Factor IX, IFN beta, AT III, A1AT, Factor VII and APC.

9. Conjugate as claimed in any of claims 5 or 8, wherein the group
—[(CR¹R²)ₘO]ₙ[CR³R⁴]ₒ—
is —CH₂CH₂OCH₂CH₂—.

10. Process for preparing a functionalized polymer comprising the step of reacting a polymer of formula III
“polymer”-(CR⁸R⁹)ₚ—Y (III)
with a compound of formula IV
Q-[(CR¹R²)ₘO]ₙ[CR³R⁴]ₒ—ONHR⁵ (IV),
wherein the symbols have the following meanings
“polymer” soluble linear or branched homopolymers or random copolymers and derivatives thereof selected from the group consisting of alkylene glycol homopolymers, alkylene glycol copolymers, polyvinyl alcohol, polyvinyl pyrrolidone, poly-1,3-dioxolane, poly-1,3,6-trioxane, ethylene/maleic anhydride copolymer, polyaminoacids and polysaccharides;
R¹, R², R³, R⁴, R⁵ hydrogen, alkyl, aryl;
m 2 to 4, wherein the residues R¹ and R² may be the same or different in the m groups CR¹R²;
n 0 to 20;
o 0 to 20, wherein in the case of n=0, o is not 0, wherein the residues R³ and R⁴ may be the same or different in the o groups CR³R⁴;
Y and Q functional groups, which are suitable to react together to give one of the following linking groups —O—, —S—, —NR⁶—, —OC(O)—, —C(O)O—, —C(G)N(R¹⁰)O—, —N(R¹¹)O—, 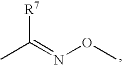
wherein one or more groups —(CR⁸R⁹)— may be replaced by W, whereby a chemically reasonable group is formed;
W O, NR¹², C(G);
G S, O, NR¹⁴;
R⁶, R⁷, R⁸, R⁹, R¹⁰, R¹¹, R¹², R¹⁴ hydrogen, alkyl, aryl;
p 0 to 20, wherein the residues R⁸ and R⁹ may be the same or different in the p groups CR⁸R⁹;
wherein the group
—(CR⁸R⁹)ₚ—Y
is covalently linked with terminal groups or centrally located groups of the “polymer”.

11. Process as claimed in claim 10, wherein Y and Q are functional groups, which are suitable to react together to give the following linking group 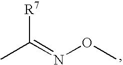
—N(R¹¹)O— or —C(G)N(R¹⁰)O—.

12. Process as claimed in claim 10 or 11, wherein the polymer is hydroxyalkyl starch, dextran or ethylene glycol homopolymer.

13. Process as claimed in any of claims 10 to 12, wherein the group
—[(CR¹R²)ₘO]ₙ[CR³R⁴]ₒ—
is —CH₂CH₂OCH₂CH₂—.

14. Functionalized polymer as obtainable by a process as claimed in any of claims 10 to 13.

15. Process for preparing a conjugate, comprising the step of reacting a functionalized polymer as claimed in any of claims 1 to 4 or 14 with a functionalized protein of formula V
“protein”-Z (V)
wherein Z is a group comprising a carbonyl group or a group which is suitable of forming a carbonyl group or another group which is reactable with the functionalized polymer, wherein Z is covalently linked with least one terminal group or least one centrally located group of the “protein”.

16. Process as claimed in claim 15, wherein a functionalized polymer as claimed in any of claims 2 to 4 is employed.

17. Process as claimed in claim 15 or 16, wherein the “protein” is selected from the group consisting of EPO, G-CSF, Factor VII, Factor IX, IFN beta, AT III, A1AT, Factor VIII and APC.

18. Process for preparing a conjugate, comprising the steps
a) reacting a polymer of formula III
“polymer”-(CR⁸R⁹)ₚ—Y (III)
with a compound of formula IV
Q-[(CR¹R²)ₘO]ₙ[CR³R⁴]ₒ—ONHR⁵ (IV),
wherein the symbols have the following meanings
“polymer” soluble linear or branched homopolymers or random copolymers and derivatives thereof selected from the group consisting of alkylene glycol homopolymers, alkylene glycol copolymers, polyvinyl alcohol, polyvinyl pyrrolidone, poly-1,3-dioxolane, poly-1,3,6-trioxane, ethylene/maleic anhydride copolymer, polyaminoacids and polysaccharides;
R¹, R², R³, R⁴, R⁵ hydrogen, alkyl, aryl;
m 2 to 4, wherein the residues R¹ and R² may be the same or different in the m groups CR¹R²;
n 0 to 20;
o 0 to 20, wherein in the case of n=0, o is not 0, wherein the residues R³ and R⁴ may be the same or different in the o groups CR³R⁴;
Y and Q functional groups, which are suitable to react together to give one of the following linking groups —O—, —S—, —NR⁶—, —OC(O)—, —C(O)O— —C(G)N(R¹⁰)O—, N(R¹¹)O—, 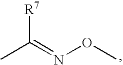
wherein one or more groups —(CR⁸R⁹)— may be replaced by W, whereby a chemically reasonable group is formed;
W O, NR², C(G);
G S, O, NR¹⁴;
R⁶, R⁷, R⁸, R⁹, R¹⁰, R¹¹, R¹², R¹⁴ hydrogen, alkyl, aryl;
p 0 to 20, wherein the residues R⁸ and R⁹ may be the same or different in the p groups CR⁸R⁹;
wherein the group
is covalently linked with least one terminal group and/or least one centrally located group of the “polymer”, wherein a functionalized polymer is obtained, and
b) reacting the functionalized polymer obtained in step a) with a functionalized protein of formula V
“protein”-Z (V)
wherein Z is a group comprising a carbonyl group or a group which is suitable of forming a carbonyl group or another group which is reactable with the functionalized polymer, wherein Z is covalently linked with least one terminal group and/or least one centrally located group of the “protein”.

19. Conjugate as obtainable by a process as claimed in any of claims 15 to 18.

20. A conjugate as claimed in any of claims 5 to 9 or 19 for use in a method for the treatment of the human or animal body.

21. A pharmaceutical composition comprising in a therapeutically effective amount a conjugate as claimed in any of claims 5 to 9 or 19.

22. The pharmaceutical composition as claimed in claim 21, further comprising at least one pharmaceutically acceptable diluent, adjuvant, or carrier.

23. Functionalized polymer as claimed in any of claims 1 to 3, wherein o is from 2 to 20.

24. Functionalized polymer as claimed in any of claims 1 to 3, wherein the group
—[(CR¹R²)ₘO]ₙ[CR³R⁴]ₒ—
is —CH₂CH₂(CH₃)CH₂CH₂.

25. Conjugate as claimed in any of claims 5 to 8, wherein o is from 2 to 20.

26. Conjugate as claimed in any of claims 5 to 8, wherein the group
—[(CR¹R²)ₘO]ₙ[CR³R⁴]ₒ—
is —CH₂CH₂(CH₃)CH₂CH₂—.

27. The process as claimed in any of claims 10 to 12, wherein o is from 2 to 20.

28. The process as claimed in any of claims 10 to 12, wherein the group
—[(CR¹R²)ₘO]ₙ[CR³R⁴]ₒ—
is —CH₂CH₂(CH₃)CH₂CH₂—.

29. Functionalized polymer as obtainable by a process as claimed in claim 27 or 28.

30. The process as claimed in claim 15 or 17, wherein a functionalized polymer as claimed in any of claims 23, 24 or 29 is reacted with the functionalized protein of formula V.

31. The process as claimed in claim 18, wherein o is from 2 to 20.

32. Conjugate obtainable by a process as claimed in claim 30.

33. A conjugate as claimed in any of claims 25, 26 or 32 for use in a method for the treatment of the human or animal body.

34. A pharmaceutical composition comprising in a therapeutically effective amount a conjugate as claimed in any of claims 25, 26 or 32.

35. The pharmaceutical composition as claimed in claim 34, further comprising at least one pharmaceutically acceptable diluent, adjuvant, or carrier.
